SEQUENCE LISTING (5 sequences)

SEQ ID NO: 1 - DNA, 79 bp, synthetic construct
  misc_feature 1..79: note = Synthetic Construct
  source 1..79: mol_type = other DNA
tttttctacacgacgctcttccgatcttgcatcataaggctctctccaagtacgatcacaccgttccaggcttagtcta

SEQ ID NO: 2 - DNA, 79 bp, synthetic construct
  misc_feature 1..79: note = Synthetic Construct
  source 1..79: mol_type = other DNA
tttttctacacgacgctcttccgatcttggtacataaggctctcatcgagtacgaatgcaacgttccaggcaagaacta

SEQ ID NO: 3 - DNA, 79 bp, synthetic construct
  misc_feature 1..79: note = Synthetic Construct
  source 1..79: mol_type = other DNA
tttttctacacgacgctcttccgatctacttcgataaggctttgttgcagtacgatgttcgagttccagcagcttacta

SEQ ID NO: 4 - DNA, 79 bp, synthetic construct
  misc_feature 1..79: note = Synthetic Construct
  source 1..79: mol_type = other DNA
tttttctacacgacgctcttccgatctaactcactaaggctcgctgaaagtacgaaagccacgttccaggttgtgacta

SEQ ID NO: 5 - DNA, 44 bp, synthetic construct
  misc_feature 1..44: note = Synthetic Construct
  misc_difference 1..12: note = n = A,T,C or G
  misc_difference 44: note = n = A,T,C or G
  source 1..44: mol_type = other DNA
nnnnnnnnnnnnttttttttttttttttttttttttttttttvn

(19) United States
(12) Patent Application Publication
(10) Pub. No.: US 2024/0060127 A1
(43) Pub. Date: Feb. 22, 2024

(54) METHODS AND SYSTEMS FOR LIGHT-CONTROLLED SURFACE PATTERNING USING PHOTOMASKS

(71) Applicant: 10x Genomics, Inc., Pleasanton, CA (US)

(72) Inventors: Denis PRISTINSKI, Dublin, CA (US); Preyas SHAH, Pleasanton, CA (US); Siyuan XING, Newark, CA (US)

(21) Appl. No.: 18/343,108

(22) Filed: Jun. 28, 2023

Related U.S. Application Data
(60) Provisional application No. 63/356,926, filed on Jun. 29, 2022.

(51) Int. Cl. C12Q 1/6874; C12Q 1/6876; G03F 7/00

(52) U.S. Cl. CPC C12Q 1/6874; C12Q 1/6876; G03F 7/0035

(57) ABSTRACT

Provided in some aspects are methods for light-controlled in situ surface patterning of a substrate using a minimal mask scheme. Systems and kits employing the methods are also disclosed. In some embodiments, a method disclosed herein comprises using photomasks and photoresist for photocontrollable hybridization and/or ligation of nucleic acid molecules, wherein photoresist removal allows hybridization and/or ligation of nucleic acid molecules at the exposed area. A large diversity of barcodes can be created in molecules on the substrate via sequential rounds of light exposure, hybridization, and ligation.

### CROSS-REFERENCE TO RELATED APPLICATION

This application claims priority to U.S. Provisional Patent Application No. 63/356,926, filed Jun. 29, 2022, entitled “METHODS AND SYSTEMS FOR LIGHT-CONTROLLED SURFACE PATTERNING USING PHOTOMASKS,” which is herein incorporated by reference in its entirety for all purposes.

### REFERENCE TO AN ELECTRONIC SEQUENCE LISTING

The contents of the electronic sequence listing (202412005500seqlist.xml; Size: 8,379 bytes; and Date of Creation: Jun. 20, 2023) are herein incorporated by reference in their entirety.

### FIELD

The present disclosure relates in some aspects to methods for manufacturing a molecular array in situ on a substrate using photomasks.

### BACKGROUND

Arrays of nucleic acids are an important tool in the biotechnology industry and related fields. These nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis, and the like. Accordingly, there is continued interest in the development of new methods for producing nucleic acid arrays in situ. Provided are methods, uses and articles of manufacture that meet such needs.

### SUMMARY

In some aspects, disclosed herein is a method for providing an array, comprising irradiating a plurality of regions on a substrate through a first photomask comprising openings that correspond to all or a subset of the plurality of regions, wherein a first oligonucleotide of at least four nucleotides in length is attached to oligonucleotide molecules in the plurality of regions to generate extended oligonucleotide molecules. In some aspects, the method further comprises irradiating the plurality of regions through the first photomask rotated relative to the substrate, or through a second photomask comprising openings that correspond to all or a subset of the plurality of regions, wherein a second oligonucleotide of at least four nucleotides in length is attached to the extended oligonucleotide molecules in the plurality of regions to generate further extended oligonucleotide molecules. In any of the preceding embodiments, the method can further comprise irradiating sub-regions of the plurality of regions through a third photomask comprising openings that correspond to the sub-regions, wherein a third oligonucleotide of at least four nucleotides in length is attached to the further extended oligonucleotide molecules in the sub-regions to generate even further extended oligonucleotide molecules. In any of the preceding embodiments, the method can provide on the substrate an array comprising oligonucleotide molecules, e.g., the extended oligonucleotide molecules, the further extended oligonucleotide molecules, and/or the even further extended oligonucleotide molecules. In any of the preceding embodiments, the method can further comprise one or more additional rounds of irradiation and oligonucleotide attachment.

In any of the embodiments herein, the oligonucleotide molecules can be directly or indirectly attached to the substrate, e.g., via a linker or spacer.

In any of the preceding embodiments, the oligonucleotide molecules on the substrate can comprise one or more common sequences. In some embodiments, the one or more common sequences comprise a common primer sequence, e.g., of between about 6 and about 45 nucleotides in length, or between about 10 and about 35 nucleotides in length. In any of the preceding embodiments, the one or more common sequences can comprise a partial primer sequence, e.g., a sequence together with a sequence of an oligonucleotide attached to an oligonucleotide molecule on the substrate forms the hybridization sequence for a primer. In any of the preceding embodiments, the oligonucleotide molecules on the substrate can comprise two or more different sequences.

In any of the preceding embodiments, the oligonucleotide molecules on the substrate can be immobilized in a plurality of regions of the substrate. In any of the preceding embodiments, the 3′ terminal nucleotides of immobilized oligonucleotide molecules can be distal to the substrate. In any of the preceding embodiments, the oligonucleotide molecules can be 5′ immobilized on the substrate. In any of the preceding embodiments, the 5′ terminal nucleotides of immobilized oligonucleotide molecules can be distal to the substrate. In any of the preceding embodiments, the oligonucleotide molecules can be 3′ immobilized on the substrate.

In any of the preceding embodiments, the plurality of regions can be arranged in rows and columns on the substrate. In any of the preceding embodiments, the first photomask and/or the second photomask can comprise openings that correspond to one or more of the rows or one or more of the columns.

In any of the preceding embodiments, the rows can be parallel to each other and/or the columns can be parallel to each other. In some embodiments, the angle between an intersecting row and column pair is 90 degrees. In any of the preceding embodiments, the plurality of regions can be arranged in a square array. In any of the preceding embodiments, the plurality of regions can be arranged in a hexagonal array.

In any of the preceding embodiments, the irradiating step using the first photomask (e.g., the same photomask) can comprise multiple cycles of irradiation and oligonucleotide attachment, and in one of the cycles the substrate is irradiated through the first photomask at a subset of the rows or a subset of the columns.

In any of the preceding embodiments, the openings in the first photomask can correspond to a column of the plurality of regions. In some embodiments, the method comprises, after irradiation and oligonucleotide attachment to the oligonucleotide molecules in a first column, translating the first photomask to a second column. In some embodiments, the first and second columns do not overlap. In any of the preceding embodiments, the first and second columns can be immediately adjacent or can be separated by one or more other columns. In any of the preceding embodiments, the method can comprise translating the first photomask and performing multiple cycles of irradiation and oligonucleotide attachment until all columns have received the first oligonucleotide, e.g., the first oligonucleotide has been attached to oligonucleotide molecules in each region in all of the columns, one column per cycle.

In any of the preceding embodiments, the first oligonucleotide can comprise a first barcode sequence. In any of the preceding embodiments, the first oligonucleotide can comprise a sequence that hybridizes to a first splint which in turn hybridizes to the oligonucleotide molecules in the plurality of regions. In some embodiments, the first splint can hybridize to all or a portion of the first barcode sequence. In any of the preceding embodiments, the first oligonucleotide can be ligated to the oligonucleotide molecules using the first splint as template to generate the extended oligonucleotide molecules. In any of the preceding embodiments, the first oligonucleotide can comprise a sequence that hybridizes to a second splint which in turn hybridizes to the second oligonucleotide. In any of the preceding embodiments, the second oligonucleotide can be ligated to the extended oligonucleotide molecules using the second splint as template to generate the further extended oligonucleotide molecules. In any of the preceding embodiments, the first barcode sequences in molecules of the first oligonucleotide can be different for different columns. In some embodiments, molecules of the first oligonucleotide delivered to the substrate in each cycle (each cycle for a different column) comprise a different barcode sequence from molecules of the first oligonucleotide in another cycle. In any of the preceding embodiments, the sequence that hybridizes to the first splint can be the same or different in molecules of the first oligonucleotide for different columns.

In any of the preceding embodiments, the irradiating step using the rotated first photomask can comprise rotating the first photomask to irradiate a first row of the plurality of regions through the openings in the first photomask. In any of the preceding embodiments, the second oligonucleotide can be attached to the extended oligonucleotide molecules in the first row. In any of the preceding embodiments, the method can comprise, after irradiation and oligonucleotide attachment to the extended oligonucleotide molecules in the first row, translating the first photomask to a second row. In some embodiments, the first and second rows do not overlap. In any of the preceding embodiments, the first and second rows can be immediately adjacent or can be separated by one or more other rows. In any of the preceding embodiments, the method can comprise translating the first photomask and performing multiple cycles of irradiation and oligonucleotide attachment until all rows have received the second oligonucleotide, e.g., the second oligonucleotide has been attached to extended oligonucleotide molecules in each region in all of the rows, one row per cycle.

In any of the preceding embodiments, the second oligonucleotide can comprise a second barcode sequence. In any of the preceding embodiments, the second oligonucleotide can comprise a sequence that hybridizes to the second splint. In some embodiments, the second splint can hybridize to all or a portion of the second barcode sequence and/or all or a portion of the first barcode sequence. In any of the preceding embodiments, the second oligonucleotide can be ligated to the extended oligonucleotide molecules using the second splint as template to generate the further extended oligonucleotide molecules. In any of the preceding embodiments, the second oligonucleotide can comprise a sequence that hybridizes to a third splint which in turn hybridizes to the third oligonucleotide. In any of the preceding embodiments, the third oligonucleotide can be ligated to the further extended oligonucleotide molecules using the third splint as template. In any of the preceding embodiments, the second barcode sequence in molecules of the second oligonucleotide can be different for different rows. In some embodiments, molecules of the second oligonucleotide delivered to the substrate in each cycle (each cycle for a different row) comprise a different barcode sequence from molecules of the second oligonucleotide in another cycle. In any of the preceding embodiments, the sequence that hybridizes to the second splint can be the same or different in molecules of the second oligonucleotide for different rows.

In any of the preceding embodiments, the openings in the second photomask can correspond to regions on the substrate arranged in one or more rows or one or more columns. In some embodiments, the first and second photomasks comprise different patterns of openings.

In any of the preceding embodiments, the irradiating step using the second photomask can comprise irradiating a first row or column of the plurality of regions through the openings in the second photomask. In any of the preceding embodiments, the second oligonucleotide can be attached to the extended oligonucleotide molecules in the first row or column, respectively.

In any of the preceding embodiments, the method can comprise, after irradiation and oligonucleotide attachment to the extended oligonucleotide molecules in the first row or column, translating the second photomask to a second row or column, respectively. In some embodiments, the first and second rows do not overlap. In some embodiments, the first and second columns do not overlap. In any of the preceding embodiments, the first and second rows or columns can be immediately adjacent or can be separated by one or more other rows or columns, respectively. In some embodiments, the method comprises translating the second photomask and performing multiple cycles of irradiation and oligonucleotide attachment until all rows or columns have received the second oligonucleotide, e.g., the second oligonucleotide has been attached to extended oligonucleotide molecules in each region in all of the rows, one column per cycle, or in all of the rows, one row per cycle.

In any of the preceding embodiments, the second oligonucleotide can comprise a second barcode sequence. In some embodiments, the first oligonucleotide is ligated to the oligonucleotide molecules using a first splint as template to generate the extended oligonucleotide molecules which comprise a sequence that hybridizes to a second splint that in turn hybridizes to the second oligonucleotide. In some embodiments, the second oligonucleotide is ligated to the extended oligonucleotide molecules using the second splint as template to generate the further extended oligonucleotide molecules.

In any of the preceding embodiments, the second oligonucleotide may comprise a sequence that hybridizes to a third splint which in turn hybridizes to the third oligonucleotide. In some embodiments, the third oligonucleotide is ligated to the further extended oligonucleotide molecules using the third splint as template. In some embodiments, the second barcode sequences in molecules of the second oligonucleotide are different for different rows or columns. In any of the preceding embodiments, the sequence that hybridizes to the second splint can be the same or different in molecules of the second oligonucleotide for different rows or columns, respectively.

In any of the preceding embodiments, the third photomask may comprise an opening that corresponds to a sub-region of each of the plurality of regions. In any of the preceding embodiments, the method can comprise, after irradiation and oligonucleotide attachment to the further extended oligonucleotide molecules in a first sub-region of each of the plurality of regions, translating the third photomask to a second sub-region of each of the plurality of regions. In some embodiments, in each of the plurality of regions, the first and second sub-regions do not overlap. In any of the preceding embodiments, the first and second sub-regions can be immediately adjacent or can be separated by one or more other sub-regions. In any of the preceding embodiments, in each of the plurality of regions, the sub-regions can be arranged in rows and columns. In any of the preceding embodiments, in each of the plurality of regions, the sub-regions can be arranged in a square array or in a hexagonal array.

In any of the preceding embodiments, the method may comprise translating the third photomask and performing multiple cycles of irradiation and oligonucleotide attachment until all sub-regions in each of the plurality of regions have received the third oligonucleotide. In some embodiments, the third oligonucleotide comprises a third barcode sequence.

In any of the preceding embodiments, the first oligonucleotide may be ligated to the oligonucleotide molecules using a first splint as template to generate the extended oligonucleotide molecules which comprise a sequence that hybridizes to a second splint that in turn hybridizes to the second oligonucleotide, the second oligonucleotide is ligated to the extended oligonucleotide molecules using the second splint as template to generate the further extended oligonucleotide molecules which comprise a sequence that hybridizes to a third splint that in turn hybridizes to the third oligonucleotide. In some embodiments, the third oligonucleotide is ligated to the further extended oligonucleotide molecules using the third splint as template to generate even further extended oligonucleotide molecules.

In any of the preceding embodiments, the third oligonucleotide may comprise a sequence that hybridizes to a fourth splint which in turn hybridizes to a fourth oligonucleotide. In any of the preceding embodiments, the fourth oligonucleotide can be ligated to the even further extended oligonucleotide molecules using the fourth splint as template.

In any of the preceding embodiments, the third barcode sequences in molecules of the third oligonucleotide can be different for different sub-regions of each of the plurality of regions. In any of the preceding embodiments, the sequences that hybridize to the third splint can be the same or different for different sub-regions of each of the plurality of regions.

In some embodiments, provided herein is a method for providing an array comprising: (a) irradiating a plurality of regions arranged in columns and rows on a substrate through a first photomask comprising periodic openings that repeat every X₁ columns and every Y₁ rows, wherein each opening corresponds to a region within a X₁×Y₁ matrix on the substrate, and wherein a first oligonucleotide of at least four nucleotides in length is attached to oligonucleotide molecules in the plurality of regions to generate extended oligonucleotide molecules; and (b) irradiating the plurality of regions through a second photomask comprising periodic openings that repeat every X₂ columns and every Y₂ rows, wherein each opening corresponds to a region within a X₂×Y₂ matrix on the substrate, and wherein a second oligonucleotide of at least four nucleotides in length is attached to the oligonucleotide molecules in the plurality of regions to generate extended oligonucleotide molecules, thereby providing on the substrate an array comprising oligonucleotide molecules. In some embodiments, X₁ and X₂ do not have a common divider. In some embodiments, Y₁ and Y₂ do not have a common divider. In any of the preceding embodiments, the method may further comprise (c) irradiating the plurality of regions through a third photomask, wherein the third photomask comprises periodic openings that repeat every X₃ columns and every Y₃ rows, and wherein a third oligonucleotide of at least four nucleotides in length is attached to the extended oligonucleotide molecules in the plurality of regions to generate further extended oligonucleotide molecules.

In some embodiments, X₁, X₂ , and X₃ do not have a common divider. In some embodiments, Y₁, Y₂ , and Y₃ do not have a common divider. In any of the preceding embodiments, X₁, Y₁, X₂, Y₂, X₃, and Y₃ can be prime numbers. In any of the preceding embodiments, X₁ and Y₁ can be the same or different, and/or X₂ and Y₂ can be the same or different, and/or X₃ and Y₃ can be the same or different. In any of the preceding embodiments, X₁, Y₁, X₂, Y₂, X₃, and Y₃ can be selected from the group consisting of three different prime numbers. In some embodiments, the plurality of regions comprise X₁×X₂×X₃×Y₁×Y₂×Y₃ different regions.

In any of the preceding embodiments, the plurality of regions can be arranged in a square array. In any of the preceding embodiments, the plurality of regions can be arranged in a hexagonal array.

In any of the preceding embodiments, the irradiating step using the first photomask can comprise multiple cycles of irradiation and oligonucleotide attachment, and in each cycle the substrate is irradiated through the first photomask at one of the regions within periodical repeats of the X₁×Y₁ matrix. In any of the preceding embodiments, the method may comprise, after irradiation and oligonucleotide attachment to the oligonucleotide molecules in a first region within the X₁×Y₁ matrix, translating the first photomask to a second region within the X₁×Y₁ matrix. In any of the preceding embodiments, X₁×Y₁ cycles of irradiation and oligonucleotide attachment can be performed, so that each region within the X₁×Y₁ matrix is irradiated. In any of the preceding embodiments, the first and second regions do not overlap. In any of the preceding embodiments, the first and second regions can be immediately adjacent or can be separated by one or more other regions within the X₁×Y₁ matrix.

In any of the preceding embodiments, the method may comprise translating the first photomask and performing X₁×Y₁ cycles of irradiation and oligonucleotide attachment until all regions within periodical repeats of the X₁×Y₁ matrix have received the first oligonucleotide.

In any of the preceding embodiments, the first oligonucleotide can comprise a first barcode sequence. In some embodiments, the first oligonucleotide comprises a sequence that hybridizes to a first splint which in turn hybridizes to the oligonucleotide molecules in the plurality of regions. In some embodiments, the first oligonucleotide is ligated to the oligonucleotide molecules using the first splint as template to generate the extended oligonucleotide molecules. In some embodiments, the first oligonucleotide comprises a sequence that hybridizes to a second splint which in turn hybridizes to the second oligonucleotide. In some embodiments, the second oligonucleotide is ligated to the extended oligonucleotide molecules using the second splint as template to generate the further extended oligonucleotide molecules.

In any of the preceding embodiments, the first barcode sequences in molecules of the first oligonucleotide can be different for different regions within the X₁×Y₁ matrix. In some embodiments, molecules of the first oligonucleotide delivered to the substrate in each cycle (each cycle for a different region within the X₁×Y₁ matrix) comprise a different barcode sequence from molecules of the first oligonucleotide in another cycle. In any of the preceding embodiments, the sequence that hybridizes to the first splint can be the same or different in molecules of the first oligonucleotide for different regions within the X₁×Y₁ matrix.

In any of the preceding embodiments, the second oligonucleotide can comprise a second barcode sequence. In some embodiments, the second oligonucleotide comprises a sequence that hybridizes to a second splint which in turn hybridizes to the first oligonucleotide in the plurality of regions. In some embodiments, wherein the second oligonucleotide is ligated to the first oligonucleotide using the second splint as template to generate the further extended oligonucleotide molecules. In some embodiments, the second oligonucleotide comprises a sequence that hybridizes to a third splint which in turn hybridizes to a third oligonucleotide. In some embodiments, the third oligonucleotide is ligated to the further extended oligonucleotide molecules using the third splint as template to generate even further extended oligonucleotide molecules.

In any of the preceding embodiments, the third oligonucleotide can comprise a third barcode sequence. In any of the preceding embodiments, the third oligonucleotide can comprise a sequence that hybridizes to the third splint. In some embodiments, the third splint can hybridize to all or a portion of the third barcode sequence and/or all or a portion of the second barcode sequence. In any of the preceding embodiments, the third oligonucleotide can be ligated to the further extended oligonucleotide molecules using the third splint as template to generate even further extended oligonucleotide molecules. In any of the preceding embodiments, the third oligonucleotide can comprise a sequence that hybridizes to a fourth splint which in turn hybridizes to a fourth oligonucleotide. In some embodiments, the fourth oligonucleotide is ligated to the even further extended oligonucleotide molecules using the fourth splint as template.

In some aspects, described herein a method for providing an array, comprising: (a) irradiating a substrate through a first photomask comprising an opening corresponding to a region of a plurality of regions on the substrate, wherein a first oligonucleotide of at least four nucleotides in length is attached to oligonucleotide molecules in the region to generate extended oligonucleotide molecules, wherein multiple cycles of the irradiation and oligonucleotide attachment are performed, one cycle for each of the plurality of regions, by translating the first photomask across the substrate until all regions have received the first oligonucleotide; and (b) irradiating the substrate through a second photomask comprising multiple openings corresponding to a set of sub-regions each of which is in one of the regions, wherein a second oligonucleotide of at least four nucleotides in length is attached to the extended oligonucleotide molecules in the set of sub-regions to generate further extended oligonucleotide molecules, wherein multiple cycles of the irradiation and oligonucleotide attachment are performed, one cycle for each set of sub-regions, by translating the second photomask across the substrate until all sub-regions of all regions have received the second oligonucleotide, thereby providing on the substrate an array comprising oligonucleotide molecules.

In any of the preceding embodiments, the method may further comprise (c) irradiating the substrate through a third photomask comprising multiple openings corresponding to a set of sub-sub-regions each of which is in one of the sub-regions, wherein a third oligonucleotide of at least four nucleotides in length is attached to the further extended oligonucleotide molecules in the set of sub-sub-regions to generate even further extended oligonucleotide molecules, wherein multiple cycles of the irradiation and oligonucleotide attachment are performed, one cycle for each set of sub-sub-regions, by translating the third photomask across the substrate until all sub-sub-regions of all sub-regions of all regions have received the third oligonucleotide.

In any of the preceding embodiments, the irradiating steps can be performed in any suitable order.

In any of the preceding embodiments, the first oligonucleotide can comprise a first barcode sequence. In any of the preceding embodiments, the first oligonucleotide can comprise a sequence that hybridizes to a first splint which in turn hybridizes to the oligonucleotide molecules in the plurality of regions. In any of the preceding embodiments, the first oligonucleotide can be ligated to the oligonucleotide molecules using the first splint as template to generate the extended oligonucleotide molecules. In any of the preceding embodiments, the first oligonucleotide can comprise a sequence that hybridizes to a second splint which in turn hybridizes to the second oligonucleotide. In any of the preceding embodiments, the second oligonucleotide can be ligated to the extended oligonucleotide molecules using the second splint as template to generate the further extended oligonucleotide molecules.

In any of the preceding embodiments, the second oligonucleotide can comprise a second barcode sequence. In any of the preceding embodiments, the second oligonucleotide can comprise a sequence that hybridizes to the second splint. In any of the preceding embodiments, the second oligonucleotide can be ligated to the extended oligonucleotide molecules using the second splint as template to generate the further extended oligonucleotide molecules. In any of the preceding embodiments, the second oligonucleotide can comprise a sequence that hybridizes to a third splint which in turn hybridizes to a third oligonucleotide. In any of the preceding embodiments, the third oligonucleotide can be ligated to the further extended oligonucleotide molecules using the third splint as template.

In any of the preceding embodiments, the third oligonucleotide can comprise a third barcode sequence. In any of the preceding embodiments, the third oligonucleotide can comprise a sequence that hybridizes to the third splint. In any of the preceding embodiments, the third oligonucleotide can be ligated to the further extended oligonucleotide molecules using the third splint as template to generate even further extended oligonucleotide molecules. In any of the preceding embodiments, the third oligonucleotide can comprise a sequence that hybridizes to a fourth splint which in turn hybridizes to a fourth oligonucleotide. In any of the preceding embodiments, the fourth oligonucleotide can be ligated to the even further extended oligonucleotide molecules using the fourth splint as template. In any of the preceding embodiments, the fourth oligonucleotide can comprise a fourth barcode sequence. In any of the preceding embodiments, the fourth splint can hybridize to all or a portion of the fourth barcode sequence and/or all or a portion of the third barcode sequence.

In any of the preceding embodiments, the first, second, third, and/or fourth oligonucleotides can comprise a unique molecular identifier (UMI). In any of the preceding embodiments, the first, second, third, and/or fourth oligonucleotides can comprise a capture sequence. In any of the preceding embodiments, the capture sequence can comprise a poly(dT) sequence. In any of the preceding embodiments, the substrate can comprise a lawn of universal oligonucleotide molecules prior to irradiation.

In any of the preceding embodiments, any one or more of the irradiating steps can comprise photolithography using a photoresist. In any of the preceding embodiments, in any one or more of the irradiating steps, the oligonucleotide molecules on the substrate, the first oligonucleotide, the second oligonucleotide, the third oligonucleotide, and/or the fourth oligonucleotide can comprise a 3′ or 5′ photo-cleavable protective group. In any of the preceding embodiments, in any one or more of the irradiating steps, the oligonucleotide molecules on the substrate, the extended oligonucleotide molecules, the further extended oligonucleotide molecules, and/or the even further extended oligonucleotide molecules can comprise a 3′ or 5′ photo-cleavable protective group. In any of the preceding embodiments, in any one or more of the irradiating steps, the oligonucleotide molecules on the substrate, the extended oligonucleotide molecules, the further extended oligonucleotide molecules, and/or the even further extended oligonucleotide molecules can be bound to a photo-cleavable polymer that blocks hybridization and/or ligation.

In any of the preceding embodiments, oligonucleotide molecules in the provided array can be located in regions which are no more than 0.2 micron, no more than 0.5 micron, no more than 1 micron, no more than 3 microns, no more than 5 microns, no more than 10 microns, no more than 15 microns, no more than 20 microns, no more than 25 microns, no more than 30 microns, no more than 35 microns, no more than 40 microns, no more than 45 microns, or no more than 50 microns in diameter.

In any of the preceding embodiments, oligonucleotide molecules in the provided array can be located in regions which are between about 0.2 micron and about 0.5 micron, between about 0.5 micron and about 1 micron, between about 1 micron and about 3 microns, between about 3 microns and about 5 microns, or between about 5 microns and about 10 microns in diameter. In any of the preceding embodiments, the average diameter of the regions can be about 0.5 micron, about 1 micron, about 2 microns, about 3 microns, about 4 microns, about 5 microns, about 6 microns, about 7 micron, about 8 microns, about 9 microns, or about 10 microns.

In any of the preceding embodiments, an oligonucleotide molecule in a region of the provided array can comprise a primer binding sequence, a combination of barcode sequences comprising the first, second, third, and/or fourth barcode sequences, a unique molecular identifier (UMI), and/or a capture sequence. In any of the preceding embodiments, oligonucleotide molecules in the same region of the provided array may comprise the same primer binding sequence, the same combination of barcode sequences, different UMIs, and the same capture sequence. In any of the preceding embodiments, oligonucleotide molecules in two or more different regions of the provided array can comprise the same primer binding sequence, different combinations of barcode sequences, and the same capture sequence. In any of the preceding embodiments, the primer binding sequence can be a universal primer binding sequence among oligonucleotide molecules of the provided array. In any of the preceding embodiments, the capture sequence can be a universal capture sequence among oligonucleotide molecules of the provided array. In any of the preceding embodiments, the combination of barcode sequences can be unique to each region of the provided array and common to oligonucleotide molecules in that region.

In any of the preceding embodiments, some or all of the oligonucleotide molecules on the substrate can be 3′ immobilized to the substrate. In any of the preceding embodiments, some or all of the oligonucleotide molecules on the substrate can be 5′ immobilized to the substrate. In some of any of the preceding embodiments, the oligonucleotide molecules, the extended oligonucleotide molecules, the further extended oligonucleotide molecules, and the even further extended oligonucleotide molecules are not provided or generated in a cell or tissue sample. In any of the preceding embodiments, the substrate can be a chip, a wafer, a die, or a slide, and the oligonucleotide molecules, the extended oligonucleotide molecules, the further extended oligonucleotide molecules, and the even further extended oligonucleotide molecules can be provided or generated in the absence of a cell or tissue sample on the substrate.

Also described herein are systems and kits capable of performing the method of any of the embodiments disclosed herein, comprising a substrate comprising a plurality of regions and one or more photomasks disclosed herein.

In some aspects, provided herein is a kit comprising the substrate, any one of more of the photomasks (e.g., the first, second, and/or third photomasks), any one or more of the oligonucleotides (e.g., the first, second, third, and/or fourth oligonucleotides), and/or any one or more of the splints (e.g., the first, second, third, and/or fourth splints) of any of the embodiments disclosed herein.

In some aspects, provided herein is a system comprising the substrate comprising oligonucleotide molecules, the first photomask, the first oligonucleotide, and the first splint of any of the embodiments disclosed herein. In some aspects, provided herein is a system comprising the substrate comprising extended oligonucleotide molecules, the first photomask or the second photomask, the second oligonucleotide, and the second splint of any of the embodiments disclosed herein. In some aspects, provided herein is a system comprising the substrate comprising further extended oligonucleotide molecules, the third photomask, the third oligonucleotide, and the third splint of any of the embodiments disclosed herein. In some aspects, provided herein is a system comprising the substrate comprising even further extended oligonucleotide molecules, a fourth photomask, the fourth oligonucleotide, and the fourth splint of any of the embodiments disclosed herein.

### BRIEF DESCRIPTION OF THE DRAWINGS

The drawings illustrate certain features and advantages of this disclosure. These embodiments are not intended to limit the scope of the appended claims in any manner.

This patent or application file contains at least one drawing executed in color. Copies of this patent or patent application publication with color drawing(s) will be provided by the Office upon request and payment of the necessary fee.

FIG. 1A shows an exemplary method for in situ array generation using photoresist.

FIG. 1B shows an exemplary barcoded molecule generated using a method described herein.

FIG. 2 is a schematic illustration of an exemplary method comprising M rounds, each of which comprise N cycles of light exposure of a region on the substrate and oligonucleotide attachment to exposed oligonucleotide molecules on the substrate.

FIGS. 3A-3C are schematic illustrations of an exemplary method using two photomasks to generate a square array. FIG. 3A shows in Round 1, a first photomask is used for column-wise incorporation of first barcode sequences (e.g., “part A” barcode sequences, or “BC-A” in FIG. 1B) into oligonucleotide molecules in the regions arranged in columns on the substrate. The regions are shown in squares as an example and can be in other suitable shapes. The first barcode sequence can be the same for regions in the same column and unique among regions in different columns. FIG. 3B shows in Round 2, the same first photomask can be rotated and used for row-wise incorporation of second barcode sequences (e.g., “part B” barcode sequences or “BC-B” in FIG. 1B) into oligonucleotide molecules in the regions arranged in rows on the substrate. The second barcode sequence can be the same for regions in the same row and unique among regions in different rows. FIG. 3C shows in Round 3, a second photomask is used for incorporation of third barcode sequences (e.g., “part C” barcode sequences or “BC-C” in FIG. 1B) into oligonucleotide molecules in a sub-region (e.g. “subpixel”) of the regions (e.g. “coarse pixels”) in FIGS. 3A-3B. In each cycle, the same fine “subpixel” relative to each “coarse pixel” can be exposed to light. For example, sub-region 6 in Column 4/Row 1 and sub-region 6 in in Column 3/Row 3 can be exposed to light in the same cycle and the same third barcode sequence can be attached to oligonucleotide molecules in these sub-regions and sub-regions 6 of other regions. The second photomask can be moved in X-Y directions to pick another subpixel for a new cycle. In the example shown in FIG. 3C, 25 cycles can be performed, and in each cycle a different third barcode sequence can be attached to oligonucleotide molecules comprising the first and second barcode sequences. Oligonucleotides comprising the barcode sequences to be incorporated can be protected (e.g., by a photo-cleavable moiety such as a photo-caged nucleobase) or not protected (e.g., the oligonucleotide molecules can be covered by a photoresist and exposed to light in a cycle). Rounds 1-3 can be performed in any suitable order.

FIGS. 4A-4C are schematic illustrations of an exemplary method using three photomasks to generate a hexagonal array. FIG. 4A shows in Round 1, a first photomask is used for column-wise incorporation of first barcode sequences (e.g., “part A” barcode sequences or “BC-A” in FIG. 1B) into oligonucleotide molecules in the regions arranged in columns on the substrate. Hexagonal regions are shown as an example and other suitable shapes can be used. The first barcode sequence can be the same for regions in the same column and unique among regions in different columns, for example as illustrated by different shades or patterns for the different columns. FIG. 4B shows in Round 2, a second photomask can be used for row-wise incorporation of second barcode sequences (e.g., “part B” barcode sequences or “BC-B” in FIG. 1B) into oligonucleotide molecules in the regions arranged in rows on the substrate. The second barcode sequence can be the same for regions in the same row and unique among regions in different rows, for example as illustrated by different shades or patterns for the different rows. FIG. 4C shows in Round 3, a third photomask is used for incorporation of third barcode sequences (e.g., “part C” barcode sequences or “BC-C” in FIG. 1B) into oligonucleotide molecules in a sub-region (“subpixel”) of the regions (“coarse pixels”) in FIGS. 4A-4B. In each cycle, the same fine “subpixel” relative to each “coarse pixel” can be exposed to light. In the example shown in FIG. 4C, 19 cycles can be performed, and in each cycle a different third barcode sequence can be attached to oligonucleotide molecules comprising the first and second barcode sequences. The spacing between two “coarse pixels” can be the same as between two “subpixels.” Rounds 1-3 can be performed in any suitable order.

FIGS. 5A-5D are illustrations of an exemplary method using three photomasks to generate a square array. FIG. 5A shows a substrate where each region can correspond to an opening in a photomask having openings arranged in sparse regular patterns in a 7×3 matrix (FIG. 5B), a 5×5 matrix (FIG. 5C), and a 3×7 matrix (FIG. 5D). FIG. 5B shows in Round 1, a first photomask comprising periodic openings (one opening per 7×3 matrix) can be used to incorporate first barcode sequences (e.g., “part A” barcode sequences, or “BC-A” in FIG. 1B) into oligonucleotide molecules in regions 0-20 in 21 cycles. With limited motion, the first photomask can be used in each cycle so that the opening (blue/shaded square) in a 7×3 matrix is positioned at region 0 for cycle 0 (for attaching first barcode sequence 0), region 1 for cycle 1 (for attaching first barcode sequence 1), and so on until region 20 for cycle 20 (for attaching first barcode sequence First barcode sequences 0-20 can be different from each other. FIG. 5C shows in Round 2, a second photomask comprising periodic openings (one opening per 5×5 matrix) can be used to incorporate second barcode sequences (e.g., “part B” barcode sequences, or “BC-B” in FIG. 1B) into oligonucleotide molecules in regions 0-24 in 25 cycles. With limited motion, the second photomask can be used in each cycle so that the opening (green/shaded square) in a 5×5 matrix is positioned at region 0 for cycle 0 (for attaching second barcode sequence 0), region 1 for cycle 1 (for attaching second barcode sequence 1), and so on until region 24 for cycle 24 (for attaching second barcode sequence 24). Second barcode sequences 0-24 can be different from each other. FIG. 5D shows in Round 3, a third photomask comprising periodic openings (one opening per 3×7 matrix) can be used to incorporate third barcode sequences (e.g., “part C” barcode sequences, or “BC-C” in FIG. 1B) into oligonucleotide molecules in regions 0-20 in 21 cycles. With limited motion, the third photomask can be used in each cycle so that the opening (red/shaded square) in a 3×7 matrix is positioned at region 0 for cycle 0 (for attaching third barcode sequence 0), region 1 for cycle 1 (for attaching third barcode sequence 1), and so on until region 20 for cycle 20 (for attaching third barcode sequence 20). Third barcode sequences 0-20 can be different from each other. Rounds 1-3 can be performed in any suitable order.

FIGS. 6A-6C are step-wise schematic illustrations of the exemplary method shown in FIGS. 5A-5D.

FIGS. 7A-7C are schematic illustrations of an exemplary method using three photomasks comprising sparse patterning to generate a hexagonal array. FIG. 7A shows in Round 1, a first photomask comprising periodic openings (one opening per 3×7 matrix; shown as black) can be used in 21 cycles to incorporate first barcode sequences into oligonucleotide molecules. FIG. 7B shows in Round 2, a second photomask comprising periodic openings (one opening per 7×3 matrix; shown as shaded) can be used in 21 cycles to incorporate second barcode sequences into oligonucleotide molecules. FIG. 7C shows in Round 3, a third photomask comprising periodic openings (one opening per 5×5 matrix; shown as striped) can be used in 25 cycles to incorporate third barcode sequences into oligonucleotide molecules. Rounds 1-3 can be performed in any suitable order.

FIGS. 8A-8C are schematic illustrations of an exemplary method using three photomasks to generate a hexagonal array. FIG. 8A shows in Round 1, a first photomask comprising periodic “fine” openings (one “fine” opening corresponding to a hexagonal region per 4×4 matrix of 16 hexagonal regions) can be used in 16 cycles to incorporate first barcode sequences into oligonucleotide molecules. FIG. 8B shows in Round 2, a second photomask comprising a “coarse” opening can be used in 17 cycles to incorporate second barcode sequences into oligonucleotide molecules. The “coarse” opening can be moved between cycles to correspond to one of the 17 rows each comprising 4 rows of hexagonal regions. FIG. 8C shows in Round 3, a third photomask comprising a “coarse” opening can be used in 15 cycles to incorporate third barcode sequences into oligonucleotide molecules. The “coarse” opening can be moved between cycles to correspond to one of the 15 columns, each column comprising 4 columns of hexagonal regions. Rounds 1-3 can be performed in any suitable order.

FIGS. 9A-9C are schematic illustrations of an exemplary method using three photomasks to generate a hexagonal array. FIG. 9A shows in Round 1, a first photomask comprising periodic openings (one opening corresponding to a hexagonal region per 2×7 matrix of 14 hexagonal regions) can be used in 14 cycles to incorporate first barcode sequences into oligonucleotide molecules. FIG. 9B shows in Round 2, a second photomask comprising larger periodic openings can be used in 18 cycles to incorporate second barcode sequences into oligonucleotide molecules. The larger periodic opening corresponds to a region comprising the 2×7 matrix of 14 hexagonal regions, and the larger periodic openings are repeated every six 2×7 matrices on the Y axis and every three 2×7 matrices on the X axis. In other words, the 2×7 matrix as a whole in FIG. 9A is a repeating unit of the 6×3 matrix in FIG. 9B. FIG. 9C shows in Round 3, a third photomask comprising even larger periodic openings can be used in 18 cycles to incorporate third barcode sequences into oligonucleotide molecules. The even larger periodic opening corresponds to a region comprising the 6×3 matrix in FIG. 9B, and the even larger periodic openings are repeated every six 6×3 matrices on the Y axis and every three 6×3 matrices on the X axis. In other words, the 6×3 matrix as a whole in FIG. 9B is a repeating unit of the 6×3 matrix in FIG. 9C. Rounds 1-3 can be performed in any suitable order.

FIGS. 10A-10D shows an exemplary method for in situ square array generation using four different sized photomasks.

FIG. 11 shows exemplary oligonucleotides with fluorophores synthesized over four rounds of photohybridization ligation in FIG. 10D.

FIGS. 12A-12B show results of analyzing a mouse brain tissue section using an exemplary array.

### DETAILED DESCRIPTION

The practice of the techniques described herein may employ, unless otherwise indicated, conventional techniques and descriptions of organic chemistry, polymer technology, molecular biology (comprising recombinant techniques), cell biology, biochemistry, and sequencing technology, which are within the skill of those who practice in the art. Such conventional techniques comprise polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3rd Ed., W. H. Freeman Pub., New York, N.Y.; and Berg et al. (2002) Biochemistry, 5^{th} Ed., W. H. Freeman Pub., New York, N.Y., all of which are herein incorporated in their entirety by reference for all purposes.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. OVERVIEW

Arrays of nucleic acids are an important tool in the biotechnology industry and related fields. These nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis, and the like.

A feature of many arrays that have been developed is that each of the distinct nucleic acids of the array is stably attached to a discrete location on the array surface, such that its position remains constant and known throughout the use of the array. Stable attachment is achieved in a number of different ways, including covalent bonding of a nucleic acid polymer to the support surface and non-covalent interaction of the nucleic acid polymer with the surface.

There are two main ways of producing nucleic acid arrays in which the immobilized nucleic acids are covalently attached to a substrate surface, i.e., via in situ synthesis in which a nucleic acid polymer is grown on the surface of the substrate in a step-wise, nucleotide-by-nucleotide fashion, or via deposition of a full, presynthesized nucleic acid/polypeptide, cDNA fragment, etc., onto the surface of the array.

While nucleic acid arrays have been manufactured using in situ synthesis techniques, applications in the field of genomics and high throughput screening have fueled the demand for precise chemistry and high fidelity of the synthesized oligonucleotides. Accordingly, there is continued interest in the development of new methods for producing nucleic acid arrays in situ. Provided are methods, uses and articles of manufacture that meet such needs.

Oligonucleotide arrays for spatial transcriptomics may be made by mechanical spotting, bead arrays, and/or in situ base-by-base synthesis of the oligonucleotides. In some cases, mechanical spotting is ideal for larger spot sizes (e.g., 30 microns in diameter or greater), since fully elaborated oligonucleotides (e.g., with a desired combination and diversity of barcodes) can be spotted in a known position with high purity and fidelity. However, methods to decrease spot sizes or features at or below 10 microns (e.g., single cell scale resolution) in diameter with sufficient throughput are lacking. In some aspects, bead arrays offer a way to increase feature density. For example, barcodes are generated by first attaching an oligonucleotide to all beads and then performing multiple rounds of split-pool ligations to generate barcodes combinatorially. However, in some aspects, bead arrays result in random barcoded bead arrays that must be decoded prior to use and each array ultimately has a unique pattern. Additionally, even monodisperse beads at the 1-micron scale may have some variability that results in a range of feature sizes with the potential for variable oligonucleotide density. Additionally, poor imaging quality of biological samples (e.g., tissue sections) deposited onto bead arrays may occur due to differences in refractive indexes of the bead array and the biological sample.

Methods for in situ generated arrays have utilized photo-cleavable protecting groups to synthesize barcode oligonucleotides one nucleotide at a time. The feature size can be highly controlled using photomasks and the generated array is known and uniform across all arrays with no decoding needed. However, the oligonucleotide fidelity for in situ arrays decreases with increasing oligo length with a ˜99% per step (i.e., per nucleotide) efficiency.

In some embodiments, provided herein are methods for the fabrication of patterned arrays (e.g., a substrate having coupled to it a plurality of polymer molecules, such as oligonucleotides) with high spatial resolution. Provided herein in some embodiments are methods and uses of light-controlled combinatorial barcode generation for in situ arrays. In some embodiments, light-controlled ligation for in situ combinatorial barcode generation with one or more photomasks is utilized.

Provided herein in some embodiments are methods and uses of photohybridization-ligation combinatorial barcode generation using a photomask and photolithography for in situ arrays. For example, a method disclosed herein may comprise translating and/or rotating one or more photomasks on a substrate for photocontrollable hybridization and/or ligation, wherein local irradiation causes degradation of a photoresist, a photo-cleavable moiety (e.g., a photo-cleavable protective group), and/or a photo-cleavable blocker (e.g., a photo-cleavable polymer) that blocks hybridization and/or ligation to oligonucleotides in the irradiated regions.

In some aspects, a method disclosed herein provides one or more advantages as compared to available arraying methods. For example, a large diversity of barcodes can be created via sequential rounds of light (e.g., UV) exposure, hybridization, and ligation, and each round may comprise sequential cycles of light exposure, hybridization, and ligation. In examples where a photoresist is used to cover oligonucleotide molecules on a substrate, sequential rounds and cycles of photoresist removal and reapplication may be used. In these examples, no protection/deprotection of oligonucleotide molecules is required for ligating oligonucleotides to molecules on the substrate. In some aspects, the feature size can be highly controlled using photomasks. In some aspects, barcode sequences at any discrete location are known in a generated array and across all arrays generated from the same wafer, with no decoding needed.

In some aspects, the uniqueness of barcodes can be achieved combinatorially with a minimal number of photomasks that require minimal manipulation (e.g., rotation and/or translation in the X- and/or Y-axis) between cycles of the same barcode round and/or between different barcoding rounds. In some aspects, the ability to use a minimal number of photomasks saves cost and time, makes the array assembly process amendable to automation, and ensures consistent light exposure between cycles of the same round and/or between different barcoding rounds. As such, arrays with consistent feature (e.g., array spots) sizes, shapes, and/or nucleotide compositions can be generated.

### II. PHOTOLITHOGRAPHY AND MOLECULAR ARRAYS

In some aspects, provided herein is a method of patterning a surface (e.g. substrate) in situ for producing an array on the surface. In some embodiments, the method comprises assembling barcode sequences on immobilized oligonucleotides, e.g., based on hybridization and/or ligation, on a surface (e.g., slide, wafer, or flow cell). In some embodiments, the in situ method uses photo-controlled hybridization/ligation to enable barcodes to be generated combinatorially, for example, in as few as three rounds of assembly.

In some aspects, hybridization and/or ligation of barcodes can be controlled using one or more photo-cleavable moieties. For example, hybridization can be blocked using a synthetic nucleotide with a photo-cleavable protecting group on a nucleobase and/or a photo-cleavable hairpin that dissociates upon cleavage. In other examples, ligation can be controlled using a photo-cleavable moiety, such as a photo-caged 3′-hydroxyl group.

In some aspects, hybridization and/or ligation of barcodes can be controlled using one or more photo-cleavable blockers such as a photo-cleavable polymer. In one aspect, a barcoded DNA array (e.g., in the form of a DNA brush) is generated via photocontrollable surface-initiated oligonucleotide hybridization. However, in place of photo-caged oligonucleotides, unmodified oligonucleotides are used. To prevent hybridization, a DNA binding polymer is introduced that binds the surface oligonucleotides, thereby forming polyplexes. Binding is typically quantitative and causes the DNA and oligonucleotides to condense into a form where it remains inaccessible. Within this polymer, photolabile groups (e.g., nitrobenzyl) are introduced either in the DNA binding polymer backbone or at each subunit. Upon exposure to UV, these photolabile bonds break and DNA is released from the polymer leaving accessible oligonucleotides suitable for hybridization. The area (e.g., sub-portion of an array or wafer) where the DNA binding polymer is to be released can be controlled by standard photolithography patterning. After a round of hybridization and ligation, the DNA binding polymer is reintroduced enabling subsequent rounds of oligo building. In some aspects, advantages of the method disclosed herein include that unmodified oligonucleotides may be used, which are less expensive, and the photo-degradation reaction of the DNA binding polymer can be less efficient. This is because not every photolabile group needs to break to release the DNA binding polymer, just enough to disrupt multivalent electrostatic interactions keeping the polymer/oligonucleotide complex together. In some aspects, the feature size can be highly controlled using photomasks and the generated array is known and uniform across all arrays with no decoding needed.

In some aspects, hybridization and/or ligation of barcodes can be controlled using a photoresist. In some embodiments, the method described herein comprises a plurality of rounds, wherein each round comprises one or more cycles. In some embodiments, each cycle within the same round comprises the following general steps: (1) selective removal of photoresist by irradiation/UV exposure; (2) ligation of oligonucleotide; (3) blocking or capping, wherein the steps are reiterated for different features. In some embodiments, each feature receives at most one oligo in a round, wherein all features are ligated to at most one part of one or more barcodes. In some embodiments, selective removal of photoresist may be achieved with translating and/or rotating one or more photomasks.

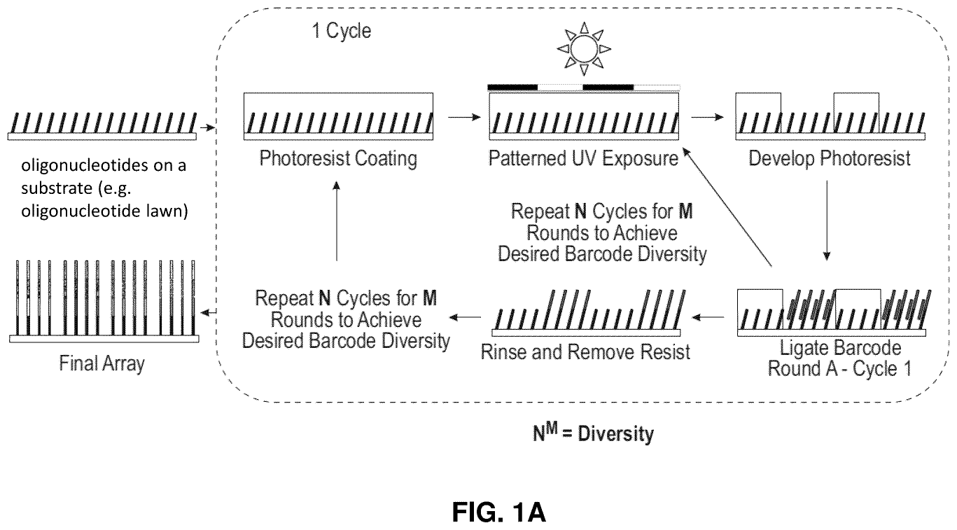

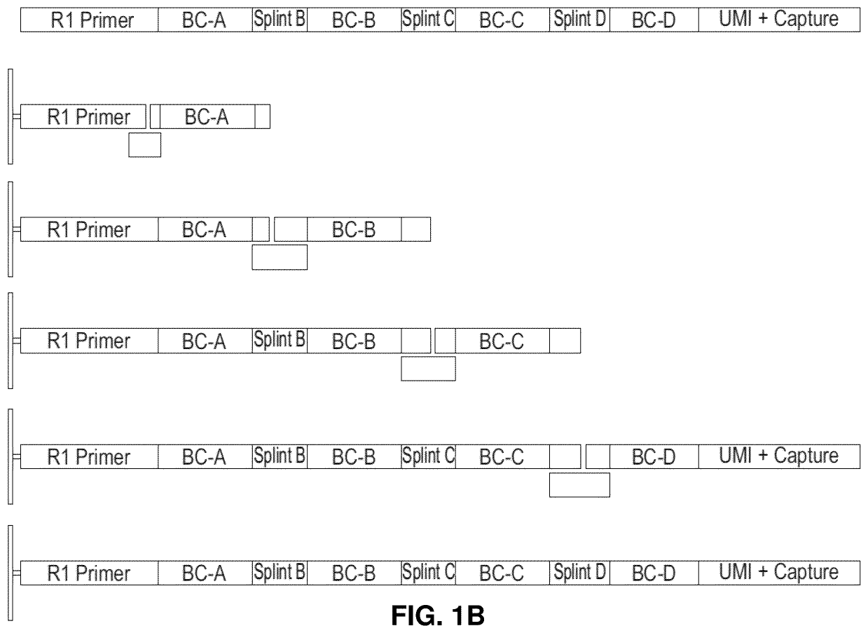

FIG. 1A provides a non-limiting example of a method for generation an in situ oligonucleotide array using hybridization/ligation and a photoresist. A photoresist coating can be applied to a substrate comprising nucleic acid molecules, such as an oligonucleotide lawn. The photoresist coating is subjected to patterned UV exposure to selectively remove photoresist at pre-determined positions on the substrate. The patterned UV exposure may be achieved by irradiating the photoresist-covered substrate with UV light through a photomask. After developing photoresist, oligonucleotides comprising a first part of a barcode are applied to the substrate, and hybridize to splints that hybridize to the oligo lawn. The oligonucleotides may be ligated to molecules of the oligo lawn at the pre-determined positions. After ligation, the photoresist coating can be optionally removed, and the irradiation-development-ligation steps are repeated for N cycles, each cycle for one or more different pre-determined positions on the substrate. After all features (e.g. defined region of oligonucleotides in the array) are ligated to at most one part of the barcode, the round may be optionally repeated. In some cases, the round is repeated M times to ligate M parts of the barcode onto the substrate, generating a nucleotide array with N^{M} sequence diversity.

In some aspects, provided herein is a method for construction of a hybridization complex or an array comprising nucleic acid molecules (e.g. oligonucleotide molecules) and complexes. FIG. 1B shows an exemplary oligonucleotide on a substrate for capturing analytes or proxies thereof which may be generated using a method disclosed herein comprising four rounds of hybridization and ligation.

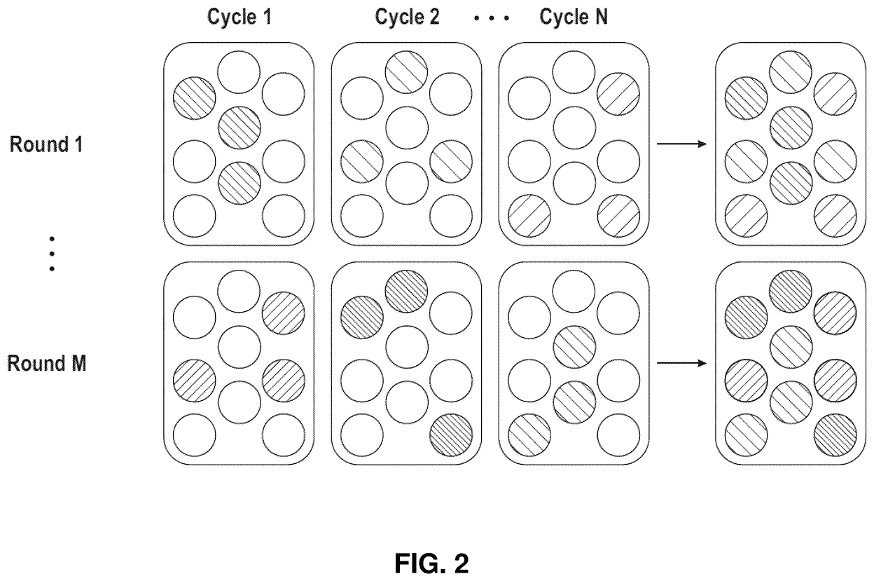

FIG. 2 further provides a schematic demonstration of an exemplary method for synthesis of barcoded oligonucleotides. In FIG. 2, features (e.g. defined regions of oligonucleotides on the array) are shown in circles. During cycle 1 of round 1, a first part of a barcode is attached to the circles marked with diagonal lines. Cycle 2 of round 1 attaches a first part of a barcode to a different set of features marked with diagonal lines, wherein the sequence of the oligonucleotide attached to the marked features may be different from those attached to the marked features in cycle 1. Upon completion of N cycles in round 1, all features are ligated with the first part of a barcode. Similarly, upon completion of N cycles in round M, all features are ligated with the M^{th} part of a barcode, and an array with N^{M} barcode diversity is generated.

### A. Photomask

In some embodiments, one or more photomasks may be used to selectively remove photoresist on the substrate. The mask is designed in such a way that the exposure sites can be selected, and thus specify the coordinates on the array where each oligonucleotide can be attached. The process can be repeated, a new mask is applied activating different sets of sites and coupling different barcodes, allowing oligonucleotide molecules to be constructed at each site. This process can be used to synthesize hundreds of thousands or millions of different oligonucleotides. In some embodiments, the substrate is irradiated through one or more patterned masks. The mask may be an opaque plate or film with transparent areas that allow light to shine through in a pre-defined pattern. In some embodiments, the transparent areas define one or more openings. The openings may be of any shape, such as round, square, or hexagonal.

The material of the photomask used herein may comprise silica with chrome in the opaque part. For example, the photomask may be transparent fused silica blanks covered with a pattern defined with a chrome metal absorbing film. The photomask may be used at various irradiation wavelengths, which include but are not limited to, 365 nm, 248 nm, and 193 nm. In some embodiments, the irradiation step herein can be performed for a duration of between about 1 minute and about 10 minutes, for example, for about 2 minutes, about 4 minutes, about 6 minutes, or about 8 minutes. In some embodiments, the irradiation can be performed at a total light dose of between about one and about ten mW/mm², for example, at about 2 mW/mm², about 4 mW/mm², about 6 mW/mm², or about 8 mW/mm². In some embodiments, the irradiation can be performed at a total light dose of between about one and about ten mW/mm² and for a duration of between about 1 minute and about 10 minutes.

In some embodiments, the photomask may comprise periodic openings. In some embodiments, the patterned mask may comprise openings that correspond to all or a subset of a plurality of regions on the substrate. In some embodiments, the photomask may comprise openings corresponding to a set of sub-regions within a region on the substrate Likewise, the plurality of regions on the substrate may be of any shape, such as round, square, or hexagonal. The plurality of regions on the substrate may be arranged in any way desired, for example, in rows and columns on the substrate, or in a hexagonal array on the substrate. In some embodiments, the rows of regions may be parallel to each other and/or the columns of regions on the substrate may be parallel to each other. In some embodiments, the angle between an intersecting row and column is 90 degrees.

After the irradiation step, the mask may be translated to a different region on the substrate, rotated to expose a different region on the substrate, or removed in order to apply a different mask to the substrate. In some embodiments, a different photomasking pattern may be used in each barcoding round. In some embodiments, the same photomasking pattern may be used in each barcoding round. Using a series of photomasks, photoresist in desired regions of the substrate may be iteratively irradiated and subsequently removed.

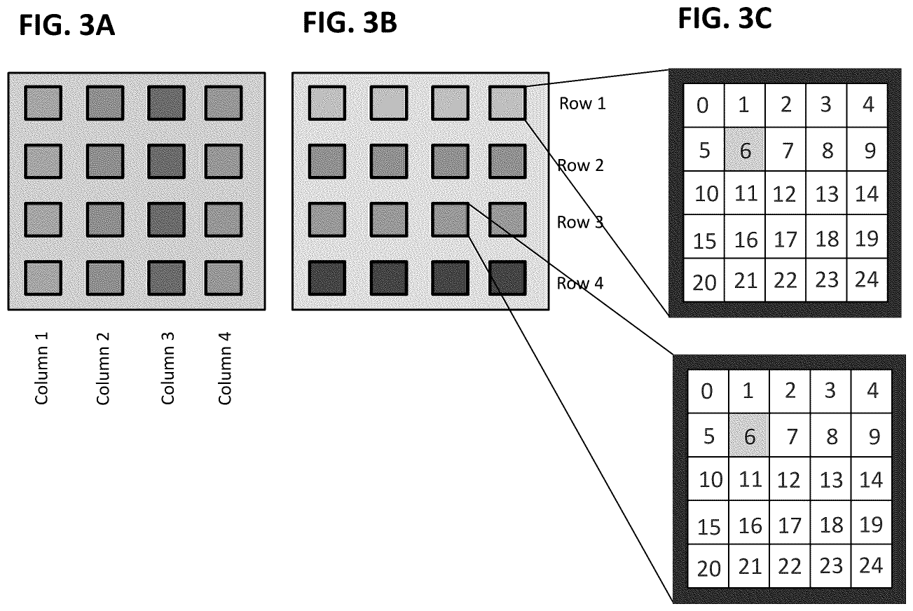

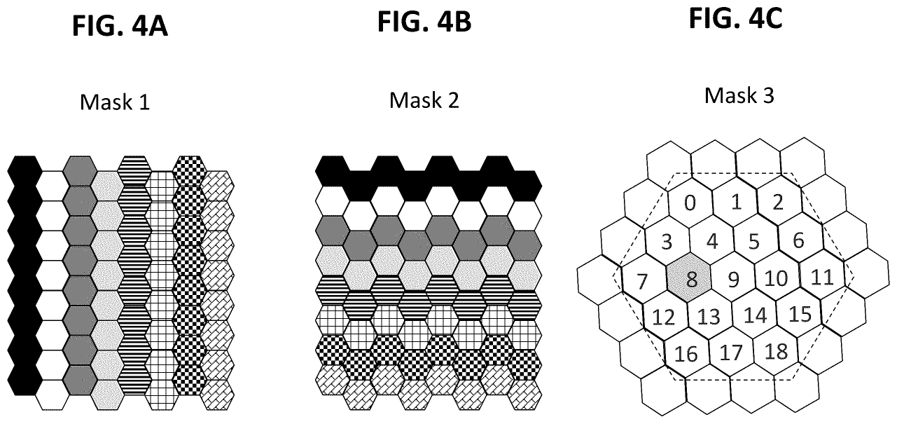

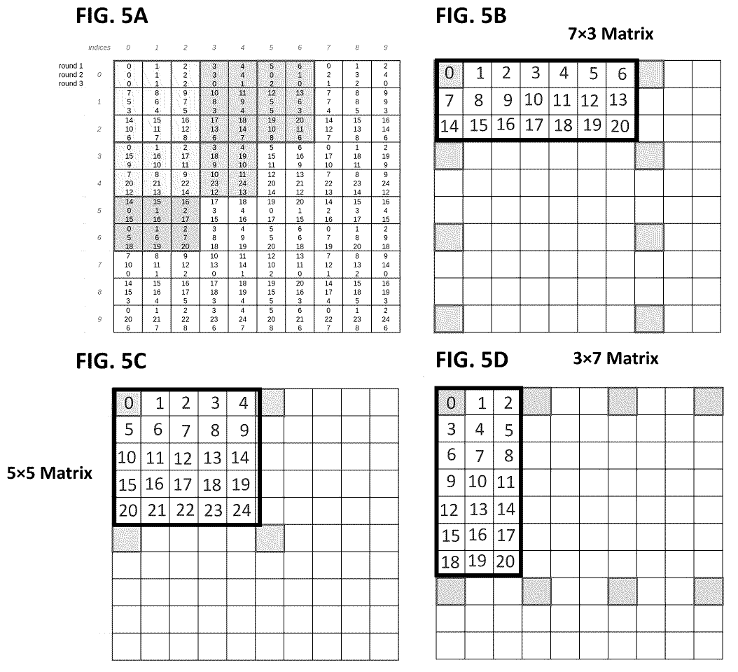

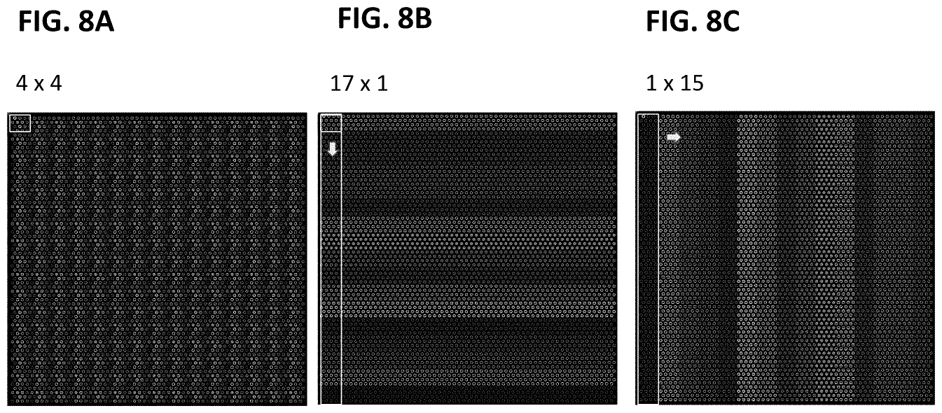

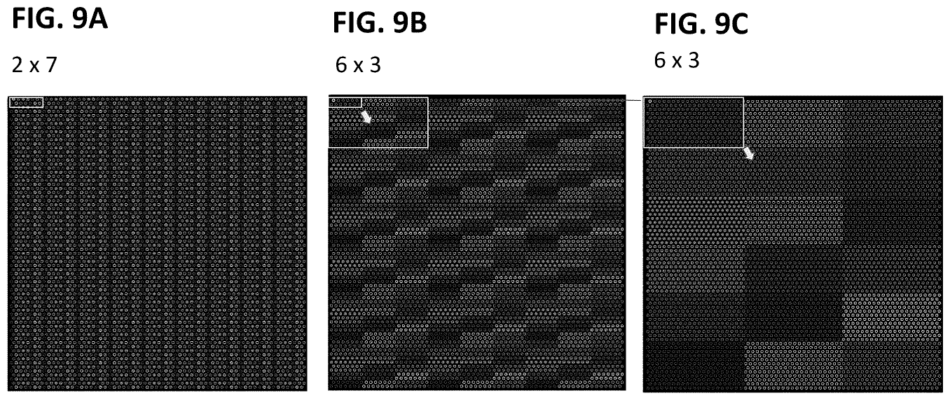

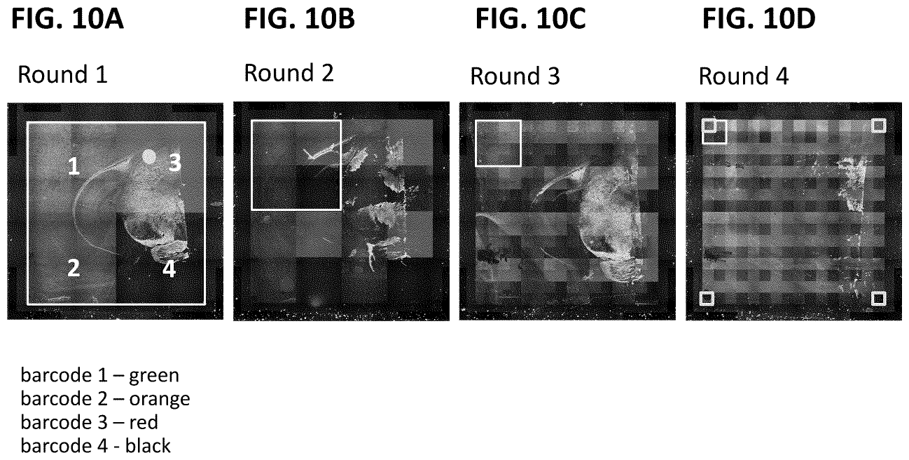

In some embodiments, a photomask can be reused in multiple cycles of the same barcoding round, as well as in multiple barcoding rounds. In some embodiments, the photomask is translated across a substrate to expose a first set of different regions of the substrate in sequential cycles of a first round, and then rotated by an angle (e.g., by 90 degrees) and reused to expose a second set of different regions of the substrate in sequential cycles of a second round, for example, as shown in FIGS. 3A-3B. The first set of regions can intersect with the second set of regions, and the overlapping regions of the first and second sets can define regions that contain combinatorially barcoded oligonucleotide molecules. In some embodiments, a photomask with a “coarse” pattern and a photomask with a “fine” pattern may be used in sequential barcoding rounds, where an opening in the “fine” pattern photomask (e.g., as shown in FIG. 3C, FIG. 4C, and FIG. 8A) is smaller than an opening in the “coarse” pattern photomask (e.g., as shown in FIGS. 3A-3B, FIGS. 4A-4B, and FIGS. 8B-8C). In some embodiments, the opening in the “coarse” pattern photomask can comprise multiple repeating units each corresponding to the opening in the “fine” pattern photomask. The opening in the “coarse” pattern photomask and/or the opening in the “fine” pattern photomask can be of any suitable shape, e.g., square, hexagonal, or round. In some embodiments, photomasks with sparse regular patternings may be used in sequential barcoding rounds (e.g., as shown in FIGS. 5A-5D, FIGS. 6A-6C, and FIGS. 7A-7C). In some embodiments, photomasks with hierarchical patternings may be used in sequential barcoding rounds (e.g., as shown in FIGS. 9A-9C and FIGS. 10A-10D). The various schemes disclosed herein, e.g., photomask rotation, “coarse-fine” patterning, and hierarchical patterning, can be used in any suitable combination and in any suitable order.

### B. Photoresist

A photoresist is a light-sensitive material used in processes (such as photolithography and photoengraving) to form a pattern on a surface. A photoresist may comprise a polymer, a sensitizer, and/or a solvent. The photoresist composition used herein is not limited to any specific proportions of the various components.

Photoresists can be classified as positive or negative. In positive photoresists, the photochemical reaction that occurs during light exposure weakens the polymer, making it more soluble to developer, so a positive pattern is achieved. In the case of negative photoresists, exposure to light causes polymerization of the photoresist, and therefore the negative photoresist remains on the surface of the substrate where it is exposed, and the developer solution removes only the unexposed areas. In some embodiments, the photoresist used herein is a positive photoresist. In some embodiments, the photoresist is removable with UV light.

The photoresist may experience changes in pH upon irradiation. In some embodiments, the photoresist comprises a photoacid generator (PAG). In some embodiments, the photoresist in all regions comprises a photoacid generator. In some embodiments, the photoresist in all regions comprises the same photoacid generator. In some embodiments, the photoresist in some of the plurality of regions comprises different photoacid generators. In some embodiments, the photoacid generator or photoacid generators irreversibly release protons upon absorption of light. Photoacid generators may be used as components of photocurable polymer formulations and chemically amplified photoresists. Examples of photoacid generators include triphenylsulfonium triflate, diphenylsulfonium triflate, diphenyliodonium nitrate, N-Hydroxynaphthalimide triflate, triarylsulfonium hexafluorophosphate salts, N-hydroxy-5-norbornene-2,3-dicarboximide perfluoro-1-butanesulfonate, bis(4-tert-butylphenyl)iodonium perfluoro-1-butanesulfonate, triphenylsulfonium trifluoromethanesulfonate, triphenylsulfonium nonafluoro-n-butanesulfonate, triphenylsulfonium perfluoro-n-octanesulfonate, and triphenylsulfonium 2-(bicyclo[2.2.1]heptan-2-yl)-1,1,2,2-tetrafluoroethanesulfonate, 4-cyclohexylphenyldiphenylsulfonium trifluoromethanesulfonate, 4-cyclohexylphenyldiphenylsulfonium nonafluoro-n-butanesulfonate, 4-cyclohexylphenyldiphenylsulfonium perfluoro-n-octanesulfonate, 4-cyclohexylphenyldiphenylsulfonium 2-(bicyclo[2.2.1]heptan-2-yl)-1,1,2,2-tetrafluoroethanesulfonate, 4-methanesulfonylphenyldiphenylsulfonium trifluoromethanesulfonate, 4-methanesulfonylphenyldiphenylsulfonium nonafluoro-n-butanesulfonate, 4-methanesulfonylphenyldiphenylsulfonium perfluoro-n-octanesulfonate, and 4-methanesulfonylphenyldiphenylsulfonium 2-(bicyclo[2.2.1]heptan-2-yl)-1,1,2,2-tetrafluoroethanesulfonate, diphenyliodonium trifluoromethanesulfonate, diphenyliodonium nonafluoro-n-butanesulfonate, diphenyliodonium perfluoro-n-octanesulfonate, diphenyliodonium 2-(bicyclo[2.2.1]heptan-2-yl)-1,1,2,2-tetrafluoroethanesulfonate, bis(4-t-butylphenyl)iodonium trifluoromethanesulfonate, bis(4-t-butylphenyl)iodonium nonafluoro-n-butanesulfonate, bis(4-t-butylphenyl)iodonium perfluoro-n-octanesulfonate, bis(4-t-butylphenyl)iodonium 2-(bicyclo[2.2.1]heptan-2-yl)-1,1,2,2-tetrafluoroethanesulfonate, 1-(4-n-butoxynaphthalen-1-yl)tetrahydrothiophenium trifluoromethanesulfonate, 1-(4-n-butoxynaphthalen-1-yl)tetrahydrothiophenium nonafluoro-n-butanesulfonate, 1-(4-n-butoxynaphthalen-1-yl)tetrahydrothiophenium perfluoro-n-octanesulfonate, 1-(4-n-butoxynaphthalen-1-yl)tetrahydrothiophenium 2-(bicyclo[2.2.1]heptan-2-yl)-1,1,2,2-tetrafluoroethanesulfonate, 1-(6-n-butoxynaphthalen-2-yl)tetrahydrothiophenium trifluoromethanesulfonate, 1-(6-n-butoxynaphthalen-2-yl)tetrahydrothiophenium nonafluoro-n-butanesulfonate, 1-(6-n-butoxynaphthalen-2-yl)tetrahydrothiophenium perfluoro-n-octanesulfonate, 1-(6-n-butoxynaphthalen-2-yl)tetrahydrothiophenium 2-(bicyclo[2.2.1]heptan-2-yl)-1,1,2,2-tetrafluoroethanesulfonate, 1-(3,5-dimethyl-4-hydroxyphenyl)tetrahydrothiophenium trifluoromethanesulfonate, 1-(3,5-dimethyl-4-hydroxyphenyl)tetrahydrothiophenium nonafluoro-n-butanesulfonate, 1-(3,5-dimethyl-4-hydroxyphenyl)tetrahydrothiophenium perfluoro-n-octanesulfonate, 1-(3,5-dimethyl-4-hydroxyphenyl)tetrahydrothiophenium 2-(bicyclo[2.2.1]heptan-2-yl)-1,1,2,2-tetrafluoroethanesulfonate N-(trifluoromethanesulfonyloxy)bicyclo[2.2.1]hept-5-ene-2,3-dicarboximide, N-(nonafluoro-n-butanesulfonyloxy)bicyclo[2.2.1]hept-5-ene-2,3-dicarboxylmide, N-(perfluoro-n-octanesulfonyloxy)bicyclo[2.2.1]hept-5-ene-2,3-dicarboxylmide, N-[2-(bicyclo[2.2.1]heptan-2-yl)-1,1,2,2-tetrafluoroethanesulfonyloxy]bicyclo[2.2.1]hept-5-ene-2,3-dicarboxylmide, N-[2-(tetracyclo[4.4.0.12,5.17,10]dodecan-3-yl)-1,1-difluoroethanesulfonyloxy]bicyclo[2.2.1]hept-5-ene-2,3-dicarboxylmide, 1,3-dioxoisoindolin-2-yl trifluoromethanesulfonate, 1,3-dioxoisoindolin-2-yl nonafluoro-n-butane sulfonate, 1,3-dioxoisoindolin-2-yl perfluoro-n-octane sulfonate, 3-dioxoisoindolin-2-yl 2-(bicyclo[2.2.1]heptan-2-yl)-1,1,2,2-tetrafluoroethanesulfonate, 3-dioxoisoindolin-2-yl N42-(tetracyclo[4.4.0.12,5.17,10]dodecan-3-yl)-1,1-difluoroethanesulfonate, 1,3-dioxo-1H-benzo[de]isoquinolin-2(3H)-yl trifluoromethanesulfonate, 1,3-dioxo-1H-benzo[de]isoquinolin-2(3H)-yl nonafluoro-n-butane sulfonate, 1,3-dioxo-1H-benzo[de]isoquinolin-2(3H)-yl perfluoro-n-octanesulfonate, 1,3-dioxo-1H-benzo[de]isoquinolin-2(3H)-yl 2-(bicyclo[2.2.1]heptan-2-yl)-1,1,2,2-tetrafluoroethanesulfonate, or 1,3-dioxo-1H-benzo[de]isoquinolin-2(3H)-yl N42-(tetracyclo [4.4.0.12,5.17,10] dodecan-3-yl)-1,1-difluoroethanesulfonate, (E)-2-(4-methoxystyryl)-4,6-bis(trichloromethyl)-1,3,5-triazine, 2-(Methoxyphenyl)-4,6-bis-(trichloromethyl)-s-triazine, 2-[2-(Furan-2-yl)ethenyl]-4,6-bis(trichloromethyl)-s-triazine, 2-[2-(5-methylfuran-2-yl]ethenyl)-4,6-bis(trichloromethyl)-s-triazine, 2-[2-(3,4-Dimethoxyphenyl)ethenyl]-4,6-bis(trichloromethyl)-s-triazine, equivalents thereof or combinations thereof. In some cases, photoacid generators capable of generating perfluoroalkanesulfonic acid having a high acid strength are used as the PAG in the formulations of the present disclosure. Such photoacid generators include, but are not limited to, photoacid generators capable of generating partially fluorinated alkane sulfonic acids, fully fluorinated alkane sulfonic acids, perfluorohexanesulfonic acid, perfluorooctanesulfonic acid, perfluoro-4-ethylcyclohexanesulfonic acid, perfluoroalkyl ether sulfonic acids, and perfluorobutanesulfonic acid. Additional examples of photoacid generators are described in U.S. Patent Pub. No. 20200384436 and U.S. Patent Pub. No. 20210017127, the contents of which are herein incorporated by reference in their entireties.

In some embodiments, a photoresist composition can form a micro or nanopattern used in lithography process for manufacturing a biomolecular array. In some embodiments, the lithography process uses a substrate material such as a wafer, e.g., a silicon-based wafer. In some embodiments, a thin (e.g., less than 1000 microns) photoresist layer is formed on a substrate, and then the substrate is optionally baked to fix the photoresist layer on the substrate. In some embodiments, the photoresist layer on the substrate is exposed to radiation. The exposed photoresist layer can be treated with a developing solution, and by dissolving and removing the exposed area of the photoresist layer, a micro or nanopattern is formed. In some embodiments, a photolithography process disclosed herein may comprise forming a photoresist layer on a substrate using a photoresist composition; selectively exposing the photoresist layer; and developing the exposed photoresist layer. In some embodiments, a photolithography process disclosed herein comprises coating a photoresist composition on a substrate and drying (soft baking) the coated substrate. In some embodiments, a photolithography process disclosed herein comprises coating with a spin coater, a bar coater, a blade coater, a curtain coater, a screen printer or the like, and/or a spray coater or the like, and any method capable of coating a photoresist composition may be used. Drying (soft baking) of the substrate may be performed under any suitable condition and may comprise, for example, an oven, a hot plate, vacuum drying and the like, but is not limited thereto. When drying, a solvent is removed from the photoresist composition, increasing adhesive strength between the substrate (e.g., wafer) and the photosensitive resist layer, and the photoresist layer may be secured on the substrate. In some embodiments, selectively exposing the photoresist layer is performed by aligning a mask on the photoresist, and exposing an area of the photoresist layer not covered by the mask to ultraviolet rays. The mask may be in contact with the photoresist layer, or may also be aligned at a certain distance from the photoresist layer. In some embodiments, a light source irradiated as a light irradiation means may comprise electromagnetic waves, extreme ultraviolet rays (EUV), from ultraviolet rays to visible rays, an electron beam, X-rays, laser rays and the like. Known means such as a high pressure mercury lamp, a xenon lamp, a carbon arc lamp, a halogen lamp, a cold cathode tube for a copier, an LED and a semiconductor laser may be used. In some embodiments, selectively exposing the photoresist layer may further comprise heating (post-exposure baking) the exposed photoresist layer after the exposure. In some embodiments, developing of the exposed photoresist layer comprises removing the exposed portion in the photoresist layer by immersing in a developing solution. Any photoresist developing methods known in the art may be used and are not limited to a rotary spray method, a paddle method, or an immersion method accompanying ultrasonic treatment. Examples of the developing solution may comprise alkali metal or alkaline earth metal hydroxides, carbonates, hydrogen carbonates, an aqueous basic solution such as an ammonia water quaternary ammonium salt may be used. For instance, an aqueous ammonia quaternary ammonium solution such as an aqueous tetramethyl ammonium solution may be used.

In some embodiments, the photoresist further comprises an acid scavenger. In some embodiments, the photoresist in all regions comprises the same acid scavenger. In some embodiments, the photoresist in some of the plurality of regions comprises different acid scavengers. In some embodiments, an acid scavenger acts to neutralize, adsorb and/or buffer acids, and may comprise a base or alkaline compound. In some embodiments, acid scavengers act to reduce the amount or concentration of protons or protonated water. In some embodiments, an acid scavenger acts to neutralize, diminish, or buffer acid produced by a photoacid generator. In some embodiments, an acid scavenger exhibits little or no stratification over time or following exposure to heat. In some embodiments, acid scavengers may be further subdivided into “organic bases” and “polymeric bases.” A polymeric base is an acid scavenger (e.g., basic unit) attached to a longer polymeric unit. A polymer is typically composed of a number of coupled or linked monomers. The monomers can be the same (to form a homopolymer) or different (to form a copolymer). In a polymeric base, at least some of the monomers act as acid scavengers. An organic base is a base which is joined to or part of a non-polymeric unit. Non-limiting examples of organic bases include, without limitation, amine compounds (e.g., primary, secondary and tertiary amines). Generally any type of acid scavenger, defined here as a traditional Lewis Base, an electron pair donor, can be used in accordance with the present disclosure. The acid scavenger may be a tertiary aliphatic amine or a hindered amine. Examples of the acid scavenger include, but are not limited to 2,2,6,6-tetramethyl-4-piperidyl stearate, 1,2,2,6,6-pentamethyl-4-piperidyl stearate, 2,2,6,6-tetramethyl-4-piperidyl benzoate, bis(2,2,6,6-tetramethyl-4-piperidyl) sebacate, bis(1,2,2,6,6-tetramethyl-4-piperidyl) sebacate, bis(1-octoxy-2,2,6,6-tetramethyl-4-piperidyl)sebacate, tetrakis(2,2,6,6-tetramethyl-4-piperidyl)-1,2,3,4-butanetetracarboxylate, tetrakis(1,2,2,6,6-pentamethyl-4-piperidyl)-1,2,3,4-butanetetracarboxylate, bis(2,2,6,6-tetramethyl-4-piperidyl) di(tridecyl)-1,2,3,4-butanetetracarboxylate, bis(1,2,2,6,6-pentamethyl-4-piperidyl) di(tridecyl)-1,2,3,4-butanetetracarboxylate, bis(1, 2,2,4, 4-pentamethyl-4-piperidyl)-2-butyl-2-(3,5-di-t-4-hydroxybenzyl)malonate, a polycondensate of 1-(2-hydroxyethyl)-2,2,6,6-tetramethyl-4-piperidinol and diethyl succinate, a polycondensate of 1,6-bis(2,2,6,6-tetramethyl-4-piperidylamino)hexane and 2,4-dichloro-6-morpholino-s-triazine, a polycondensate of 1,6-bis(2,2,6,6-tetramethyl-4-piperidylamino)hexane and 2,4-dichloro-6-t-octylamino-s-triazine, 1.5.8.12-tetrakis[2,4-bis(N-butyl-N-(2,2,6,6-tetramethyl-4-piperidyl)amino)-s-triazin-6-yl]-1.5.8.12-tetraazadodecane, 1,5,8,12-tetrakis[2,4-bis(N-butyl-N-(1,2,2,6,6-pentamethyl-4-piperidyl)amino)-s-triazin-6-yl]-1,5,8-12-tetraazadodecane, 1,6,1 1-tris[2,4-bis(N-butyl-N-(2,2,6,6-tetramethyl-4-piperidyl)amino)-s-triazin-6-yl]aminoundecane, and 1,6,1 1-tris[2,4-bis(N-butyl-N-(1,2,2,6,6-pentamethyl-4-piperidyl)amino)-s-triazin-6-yl]aminoundecane.

In some embodiments, the photoresist comprises a quencher, such as a base quencher. The quencher that may be used in the photoresist composition may comprise a weak base that scavenges trace acids, while not having an excessive impact on the performance of the photoresist. Illustrative examples of quenchers that can be employed include, but are not limited to: aliphatic amines, aromatic amines, carboxylates, hydroxides, or combinations thereof and the like. Base quenchers may comprise aliphatic amines, aromatic amines, carboxylates, hydroxides, or combinations thereof. Examples of base quenchers include but are not limited to, trioctylamine, 1,8-diazabicyclo[5.4.0]undec-7-ene (DBU), 1-piperidineethanol (1PE), tetrabutylammonium hydroxide (TBAH), dimethylamino pyridine, 7-diethylamino-4-methyl coumarin (Coumarin 1), tertiary amines, sterically hindered diamine and guanidine bases such as 1,8-bis(dimethylamino)naphthalene (PROTON SPONGE), berberine, or polymeric amines such as in the PLURONIC or TETRONIC series commercially available from BASF. In some embodiments, the photoresist in all regions comprises the same base quencher. In some embodiments, the photoresist in some of the plurality of regions comprise different base quenchers.

In some embodiments, the photoresist further comprises a photosensitizer. A photosensitizer is a molecule that produces a chemical change in another molecule in a photochemical process. Photosensitizers are commonly used in polymer chemistry in reactions such as photopolymerization, photocros slinking, and photodegradation. Photosensitizers generally act by absorbing ultraviolet or visible region of electromagnetic radiation and transferring it to adjacent molecules. In some embodiments, photosensitizer shifts the photo sensitivity to a longer wavelength of electromagnetic radiation. The sensitizer, also called a photosensitizer, is capable of activating the photoacid generator (PAG) at, for example, a longer wavelength of light in accordance with an aspect of the present disclosure. In some embodiments, the concentration of the sensitizer is greater than that of the PAG, such as 1.1 times to 5 times greater, for example, 1.1 times to 3 times greater the concentration of PAG. Examples of photosensitizer may include anthracene, N-alkyl carbazole, benzo[a]phenoxazine, and thioxanthone compounds. Exemplary sensitizers suitable for use in the methods disclosed herein include but are not limited to, isopropylthioxanthone (ITX), and 10H-phenoxazine (PhX). In some embodiments, the photoresist in the first and the second region comprises the same photosensitizer. In some embodiments, the photoresist in the first and the second region comprises different photosensitizers. Additional examples of photosensitizers include anthracenes { anthracene, 9,10-dibutoxyanthracene, 9,10-dimethoxyanthracene, 2-ethyl-9,10-dimethoxyanthracene, 2-tert-butyl-9,10-dimethoxyanthracene, 2,3-dimethyl-9,10-dimethoxyanthracene, 9-methoxy-10-methylanthracene, 9,10-diethoxyanthracene, 2-ethyl-9,10-diethoxyanthracene, 2-tert-butyl-9,10-diethoxyanthracene, 2,3-dimethyl-9,10-diethoxyanthracene, 9-ethoxy-10-methylanthracene, 9,10-dipropoxyanthracene, 2-ethyl-9,10-dipropoxyanthracene, 2-tert-butyl-9,10-dipropoxyanthracene, 2,3-dimethyl-9,10-dipropoxyanthracene, 9-isopropoxy-10-methylanthracene, 9,10-dibenzyloxyanthracene, 2-ethyl-9,10-dibenzyloxyanthracene, 2-tert-9,10-dibenzyloxyanthracene, 2,3-dimethyl-9,10-dibenzyloxyanthracene, 9-benzyloxy-10-methylanthracene, 9,10-di-α-methylbenzyloxyanthracene, 2-ethyl-9,10-di-α-methylbenzyloxyanthracene, 2-tert-9,10-di-α-methylbenzyloxyanthracene, 2,3-dimethyl-9,10-di-α-methylbenzyloxyanthracene, 9-(α-methylbenzyloxy)-10-methylanthracene, 9,10-diphenylanthracene, 9-methoxyanthracene, 9-ethoxyanthracene, 9-methylanthracene, 9-bromoanthracene, 9-methylthioanthracene, 9-ethylthioanthracene, and the like}; pyrene; 1,2-benzanthracene; perylene; tetracene; coronene; thioxanthones {thioxanthone, 2-methylthioxanthone, 2-ethylthioxanthone, 2-chlorothioxanthone, 2-isopropylthioxanthone, 2,4-diethylthioxanthone, and the like }; phenothiazine; xanthone; naphthalenes {1-naphthol, 2-naphthol, 1-methoxynaphthalene, 2-methoxynaphthalene, 1,4-dihydroxynaphthalene, 1,5-dihydroxynaphthalene, 1,6-dihydroxynaphthalene, 2,7-dihydroxynaphthalene, 2,7-dimethoxynaphthalene, 1,1′-thiobis(2-naphthol), 1,1′-bis-(2-naphthol), 4-methoxy-1-naphthol, and the like }; ketones {dimethoxyacetophenone, diethoxyacetophenone, 2-hydroxy-2-methyl-1-phenylpropan-1-one, 4′-isopropyl-2-hydroxy-2-methylpropiophenone, 2-hydroxymethyl-2-methylpropiophenone, 2,2-dimethoxy-1,2-diphenylethan-1-one, p-dimethylaminoacetophenone, p-tert-butyldichloroacetophenone, p-tert-butyltrichloroacetophenone, p-azidobenzalacetophenone, 1-hydroxycyclohexyl phenyl ketone, benzoin, benzoin methyl ether, benzoin ethyl ether, benzoin isopropyl ether, benzoin n-dibutyl ether, benzoin isobutyl ether, 1-[4-(2-hydroxyethoxy)phenyl]-2-hydroxy-2-methyl-1-propan-1-one, benzophenone, methyl o-benzoylbenzoate, Michler's ketone, 4,4′-bi sdiethylaminobenzophenone, 4,4′-dichlorobenzophenone, 4-benzoyl-4′-methyldiphenylsulfide, and the like}; carbazoles {N-phenylcarbazole, N-ethylcarbazole, poly-N-vinylcarbazole, N-glycidylcarbazole, and the like}; chrysenes {1,4-dimethoxychrysene, 1,4-diethoxychrysene, 1,4-dipropoxychrysene, 1,4-dibenzyloxychrysene, 1,4-di-α-methylbenzyloxychrysene, and the like}; and phenanthrenes 19-hydroxyphenanthrene, 9-methoxyphenanthrene, 9-ethoxyphenanthrene, 9-benzyloxyphenanthrene, 9,10-dimethoxyphenanthrene, 9,10-diethoxyphenanthrene, 9,10-dipropoxyphenanthrene, 9,10-dibenzyloxyphenanthrene, 9,10-di-α-methylbenzyloxyphenanthrene, 9-hydroxy-10-methoxyphenanthrene, 9-hydroxy-10-ethoxyphenanthrene and are described in U.S. Patent Pub. No. 20200384436, the content of which is herein incorporated by reference in its entirety.

In some embodiments, the photoresist further comprises a matrix. The matrix generally refers to polymeric materials that may provide sufficient adhesion to the substrate when the photoresist formulation is applied to the top surface of the substrate, and may form a substantially uniform film when dissolved in a solvent and spread on top of a substrate. Examples of a matrix may include, but are not limited to, polyester, polyimide, polyethylene naphthalate (PEN), polyvinyl chloride (PVC), polymethylmethacrylate (PMMA), polyglycidalmethacrylate (PGMA), and polycarbonate, or a combination thereof. The matrix may be chosen based on the wavelength of the radiation used for the generation of acid when using the photoresist formulation, the adhesion properties of the matrix to the top surface of the substrate, the compatibility of the matrix to other components of the formulation, and the ease of removable or degradation (if needed) after use. In some embodiments, the photoresist in all regions comprises the same matrix. In some embodiments, the photoresist in some of the plurality of regions comprises different matrices.

In some embodiments, the photoresist further comprises a surfactant. Surfactants may be used to improve coating uniformity, and may include ionic, non-ionic, monomeric, oligomeric, and polymeric species, or combinations thereof. Examples of possible surfactants include fluorine-containing surfactants such as the FLUORAD series available from 3M Company in St. Paul, Minn., and siloxane-containing surfactants such as the SILWET series available from Union Carbide Corporation in Danbury, Conn. In some embodiments, the photoresist in all regions comprises the same surfactant. In some embodiments, the photoresist in some of the plurality of regions comprises different surfactants.

In some embodiments, the photoresist further comprises a casting solvent. A casting solvent may be used so that the photoresist may be applied evenly on the substrate surface to provide a defect-free coating. Examples of suitable casting solvents may include ethers, glycol ethers, aromatic hydrocarbons, ketones, esters, ethyl lactate, y-butyrolactone, cyclohexanone, ethoxyethylpropionate (EEP), a combination of EEP and gamma-butyrolactone (GBL), and propylene glycol methyl ether acetate (PGMEA), and combinations thereof. In some embodiments, the photoresist in all regions comprises the same casting solvent. In some embodiments, the photoresist in some of the plurality of regions comprises different casting solvents. . In some embodiments, the solvent may comprise but is not limited to acetone, methyl ethyl ketone, methyl isobutyl ketone, methyl cellosolve, ethyl cellosolve, tetrahydrofuran, 1,4-dioxane, ethylene glycol dimethyl ether, ethylene glycol diethyl ether, propylene glycol dimethyl ether, propylene glycol diethyl ether, diethylene glycol dimethyl ether, diethylene glycol diethyl ether, diethylene glycol methyl ethyl ether, chloroform, methylene chloride, 1,2-dichloroethane, 1,1,1-trichloroethane, 1,1,2-trichloroethane, 1,1,2-trichloroethene, hexane, heptane, octane, cyclohexane, benzene, toluene, xylene, methanol, ethanol, isopropanol, propanol, butanol, t-butanol, 2-ethoxypropanol, 2-methoxypropanol, 3-methoxybutanol, cyclohexanone, cyclopentanone, propylene glycol methyl ether acetate, propylene glycol ethyl ether acetate, 3-methoxybutyl acetate, ethyl 3-ethoxypropionate, ethyl cellosolve acetate, methyl cellosolve acetate, butyl acetate, propylene glycol monomethyl ether, or dipropylene glycol monomethyl ether, or any combination thereof. In some embodiments, the solvent may comprise any one or more of those selected from the group consisting of ketones such as y-butyrolactone, 1,3-dimethyl-imidazolidinone, methyl ethyl ketone, cyclohexanone, cyclopentanone and 4-hydroxy-4-methyl-2-pentanone; aromatic hydrocarbons such as toluene, xylene and tetramethylbenzene; glycol ethers (cellosolve) such as ethylene glycol monoethyl ether, ethylene glycol monomethyl ether, ethylene glycol monobutyl ether, diethylene glycol monoethyl ether, diethylene glycol monomethyl ether, diethylene glycol monobutyl ether, propylene glycol monomethyl ether, propylene glycol monoethyl ether, dipropylene glycol diethyl ether and triethylene glycol monoethyl ether; ethyl acetate, butyl acetate, ethylene glycol monoethyl ether acetate, ethylene glycol monobutyl ether acetate, diethylene glycol monoethyl ether acetate, dipropylene glycol monomethyl ether acetate, propylene glycol monomethyl ether acetate (PGMEA), ethanol, propanol, ethylene glycol, propylene glycol, carbitol, dimethylacetamide (DMAc), N,N-diethylacetamide, dimethylformamide (DMF), diethylformamide (DEF), N,N-dimethylacetamide (DMAc), N-methylpyrrolidone (NMP), N-ethylpyrrolidone (NEP), 1,3-dimethyl-2-imidazolidinone, N,N-dimethylmethoxyacetamide, dimethyl sulfoxide, pyridine, dimethyl sulfone, hexamethylphosphoramide, tetramethylurea, N-methylcaprolactam, tetrahydrofuran, m-dioxane, p-dioxane, 1,2-dimethoxyethane, bis(2-methoxyethyl)ether, 1,2-bis(2-methoxyethoxy)ethane, bis[2-(2-methoxyethoxy)]ether, and mixtures thereof. Examples of solvents for use in photoresists are described in U.S. Patent Pub. No. 20200384436 and U.S. Patent Pub. No. 20210017127, the contents of which are herein incorporated by reference in their entireties.

In some embodiments of aspects provided herein, the photoresist composition comprises: the photoacid generator: about 1-10% (e.g., about 2-5%) by weight; the photosensitizer: about about 1-10% (e.g., 2-5%) by weight; an acid scavenger: about 0.1-0.5% by weight; a matrix: about 2.5-4.5% by weight; and a solvent. In some embodiments of aspects provided herein, the photoresist composition comprises: the photoacid generator: about 2.5-4.5% by weight; the photosensitizer: about 2.5-4.5% by weight; the acid scavenger: about 0.15-0.35% by weight; the matrix: about 3.0-4.0% by weight; and the solvent. In some embodiments of aspects provided herein, weight percentage of the photosensitizer is substantially the same as weight percentage of the photoacid generator. In some embodiments of aspects provided herein, the weight percentage of the photosensitizer is the same as the weight percentage of the photoacid generator. Suitable photoresist compositions are described, for example, in U.S. Patent Pub. No. 20200384436, the content of which is herein incorporated by reference in its entirety.

Methods of applying photoresist to the substrate include, but are not limited to, dipping, spreading, spraying, or any combination thereof. In some embodiments, the photoresist is applied via spin coating, thereby forming a photoresist layer on the substrate.

In some embodiments, the photoresist is in direct contact with the oligonucleotides on the substrate. In some embodiments, the oligonucleotide molecules on the substrate are embedded in the photoresist. In some embodiments, the photoresist is not in direct contact with the oligonucleotides. In some embodiments, oligonucleotide molecules on the substrate are embedded in an underlayer that is underneath the photoresist. For example, oligonucleotide molecules on the substrate may be embedded in a soluble polymer underlayer (e.g., a soluble polyimide underlayer (XU-218)), and the photoresist forms a photoresist layer on top of the underlayer.

In some embodiments, the photoresist may be removed and re-applied for one or more times. For example, the photoresist may be stripped from the substrate and/or the oligonucleotides ligated to the substrate. Removal of photoresist can be accomplished with various degrees of effectiveness. In some embodiments, the photoresist is completely removed from the substrate and/or the oligonucleotides ligated to the substrate before re-application. Methods of removing photoresist may include, but are not limited to, using organic solvent mixtures, using liquid chemicals, exposure to a plasma environment, or other dry techniques such as UV/O₃ exposure. In some embodiments, the photoresist is stripped using organic solvent.

### C. Molecular Arrays

In some aspects, the method provided herein comprises attaching oligonucleotides (e.g. a barcode or an oligonucleotide comprising a barcode) to a substrate. Oligonucleotides may be attached to the substrate according to the methods set forth in U.S. Pat. Nos. 6,737,236, 7,259,258, 7,375,234, 7,309,593, 7,427,678, 5,610,287, 5,807,522, 5,837,860, and 5,472,881; U.S. Patent Application Publication Nos. 2008/0280773, 2011/0059865, and 2011/0059865; Shalon et al. (1996) Genome Research, 639-645; Rogers et al. (1999) Analytical Biochemistry 266, 23-30; Stimpson et al. (1995) Proc. Natl. Acad. Sci. USA 92, 6379-6383; Beattie et al. (1995) Clin. Chem. 45, 700-706; Lamture et al. (1994) Nucleic Acids Research 22, 2121-2125; Beier et al. (1999) Nucleic Acids Research 27, 1970-1977; Joos et al. (1997) Analytical Biochemistry 247, 96-101; Nikiforov et al. (1995) Analytical Biochemistry 227, 201-209; Timofeev et al. (1996) Nucleic Acids Research 24, 3142-3148; Chrisey et al. (1996) Nucleic Acids Research 24, 3031-3039; Guo et al. (1994) Nucleic Acids Research 22, 5456-5465; Running and Urdea (1990) BioTechniques 8, 276-279; Fahy et al. (1993) Nucleic Acids Research 21, 1819-1826; Zhang et al. (1991) 19, 3929-3933; and Rogers et al. (1997) Gene Therapy 4, 1387-1392. The entire contents of each of the foregoing documents are incorporated herein by reference.

In some embodiments, oligonucleotides may be immobilized by spotting (e.g., DNA printing) on a substrate with reactive surface chemistry, such as a polymer (e.g., a hydrophilic polymer) containing epoxy reactive groups. In some embodiments, the polymer comprises a passivating polymer. In some embodiments, the polymer comprises a photoreactive group for attachment to the substrate (such as a glass slide). In some embodiments, the oligonucleotides may be immobilized in a DNA printing buffer, optionally wherein the printing buffer comprises a surfactant such as sarcosyl (e.g., a buffer containing sodium phosphate and about 0.06% sarcosyl). In some embodiments, after immobilization of the oligonucleotides, one or more wash and/or blocking steps are performed. Blocking steps can comprise contacting the substrate with a solution that deactivates or blocks unreacted functional groups on the substrate surface. In one example, the blocking buffer can comprise ethanolamine (e.g., to deactivate epoxy silane or other epoxy reactive functional groups).

Arrays can be prepared by a variety of methods. In some embodiments, arrays are prepared through the synthesis (e.g., in situ synthesis) of oligonucleotides on the array, or by jet printing or lithography. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection. Many of these methods are known in the art, and are described e.g., in Miller et al., “Basic concepts of microarrays and potential applications in clinical microbiology.” Clinical microbiology reviews 22.4 (2009): 611-633; US201314111482A; U.S. Pat. No. 9,593,365B2; US2019203275; and WO2018091676, the contents of each of which are incorporated herein by reference in their entirety.

In some embodiments, a substrate comprising an array of molecules is provided, e.g., in the form of a lawn of polymers (e.g., oligonucleotides) on the substrate (e.g. a surface) in a pattern. Examples of polymers on an array may include, but are not limited to, nucleic acids, peptides, phospholipids, polysaccharides, heteromacromolecules in which a known drug is covalently bound to any of the above, polyurethanes, polyesters, polycarbonates, polyureas, polyamides, polyethyleneimines, polyarylene sulfides, polysiloxanes, polyimides, and polyacetates. The molecules occupying different features (e.g. specific defined regions) of an array typically differ from one another, although some redundancy in which the same polymer occupies multiple features can be useful as a control. For example, in a nucleic acid array, the nucleic acid molecules within the same feature are typically the same, whereas nucleic acid molecules occupying different features are mostly different from one another.

In some examples, the molecules on the array may be nucleic acids. The nucleic acid molecule can be single-stranded or double-stranded. Nucleic acid molecules on an array may be DNA or RNA. The DNA may be single-stranded or double-stranded. The DNA may include, but are not limited to, mitochondrial DNA, cell-free DNA, complementary DNA (cDNA), genomic DNA, plasmid DNA, cosmid DNA, bacterial artificial chromosome (BAC), or yeast artificial chromosome (YAC). The RNA may include, but are not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long non-coding RNAs (lncRNAs).

In some embodiments, the molecules on an array comprise oligonucleotide barcodes. A barcode sequence can be of varied length. In some embodiments, the barcode sequence is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, or about 70 nucleotides in length. In some embodiments, the barcode sequence is between about 4 and about 25 nucleotides in length. In some embodiments, the barcode sequences is between about 10 and about 50 nucleotides in length. The nucleotides can be completely contiguous, i.e., in a single contiguous stretch of adjacent nucleotides, or they can be separated into two or more separate subsequences that are separated by 1 or more nucleotides. In some embodiments, the barcode sequence can be about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at least about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or shorter.

The oligonucleotide can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotides sequences, one or more random nucleic acid and/or amino acid sequences, and/or one or more synthetic nucleic acid and/or amino acid sequences.

In some embodiments, the UMI is a nucleic acid sequence that does not substantially hybridize to analyte nucleic acid molecules in a biological sample. In some embodiments, the UMI has less than 90% sequence identity (e.g., less than 80%, 70%, 60%, 50%, or less than 40% sequence identity) to the nucleic acid sequences across a substantial part (e.g., 80% or more) of the nucleic acid molecules in the biological sample.

The UMI can include from about 6 to about 20 or more nucleotides within the sequence of capture probes, e.g., barcoded oligonucleotides in an array generated using a method disclosed herein. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter. These nucleotides can be contiguous, i.e., in a single stretch of adjacent nucleotides, or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, a UMI is attached to other parts of the nucleotide in a reversible or irreversible manner. In some embodiments, a UMI is added to, for example, a fragment of a DNA or RNA sample before, during, and/or after sequencing of the analyte. In some embodiments, a UMI allows for identification and/or quantification of individual sequencing-reads. In some embodiments, a UMI is a used as a fluorescent barcode for which fluorescently labeled oligonucleotide probes hybridize to the UMI.

In some embodiments, a method provided herein further comprises a step of providing the substrate. A wide variety of different substrates can be used for the foregoing purposes. In general, a substrate can be any suitable support material. The substrate may comprise materials of one or more of the IUPAC Groups 4, 6, 11, 12, 13, 14, and 15 elements, plastic material, silicon dioxide, glass, fused silica, mica, ceramic, or metals deposited on the aforementioned substrates. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, quartz, metals, inorganic glasses, optical fiber bundles, and polymers, such as polystyrene, cyclic olefin copolymers (COCs), cyclic olefin polymers (COPs), polypropylene, polyethylene and polycarbonate.

A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide). In some embodiments, the substrate is a glass substrate.

In some embodiments, the surface of the substrate is coated. In some embodiments, the surface of the substrate is coated with a photoresist. In some embodiments, the method described herein comprises applying the photoresist to the substrate. In some embodiments, the substrate comprises a pattern of oligonucleotide molecules on the substrate prior to photoresist(s) being applied to the substrate. In some embodiments, the substrate does not comprise a pattern of oligonucleotide molecules on the substrate prior to photoresist(s) being applied to the substrate. In some embodiments where the substrate does not comprise oligonucleotide molecules prior to the application of photoresist(s), the substrate comprises a plurality of functional groups. In some embodiments, the plurality of functional groups of the substrate are not protected, for example, by photo-sensitive groups, moieties, or molecules. In some embodiments, the plurality of functional groups are aldehyde groups. In some embodiments, the plurality of functional groups of the substrate are click chemistry groups. The click chemistry group may be capable of various chemical reactions, which include but are not limited to, a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, or a Cu(I)-catalyzed azide-alkyne cycloaddition (CuAAC) reaction. In some embodiments, the plurality of functional groups on the substrate react with functional groups in functionalized oligonucleotide molecules. In some embodiments, the functional groups in the functionalized oligonucleotide molecules are amino groups. In some embodiments, the functionalized oligonucleotide molecules are 5′ amine-terminated.

In some embodiments, during the contact between the functionalized oligonucleotide molecules and the functional groups on the substrate, the substrate is heated to dryness. In some embodiments, after the contact between the functionalized oligonucleotide molecules and the functional groups on the substrate, the substrate is heated to dryness. In some embodiments according to any one of the methods described herein, the method further comprises blocking unreacted functional groups of the substrate. In some embodiments, the method comprises rendering the reaction between functional groups of the substrate and the functionalized oligonucleotide molecules irreversible. For example, the aldehyde groups of the substrate are reacted with 5′ amino groups of the functionalized oligonucleotide molecules, and the substrate is contacted with a reagent to block unreacted aldehyde groups and render the reaction irreversible. In some embodiment, the reagent is a reductive agent. In some embodiment, the reagent is sodium borohydride.

### III. HYBRIDIZATION/LIGATION

The nucleotide barcode parts described herein may be linked via phosphodiester bonds. The nucleotide barcode parts may also be linked via non-natural oligonucleotide linkages such as methylphosphonate or phosphorothioate bonds, via non-natural biocompatible linkages such as click-chemistry, via enzymatic biosynthesis of nucleic acid polymers such as by polymerase or transcriptase, or a combination thereof. Ligation may be achieved using methods that include, but are not limited to, primer extension, hybridization ligation, and chemical ligation. In some embodiments, the oligonucleotide comprising the barcode sequence is hybridized to a splint which is in turn hybridized to an oligonucleotide molecule in the unmasked region. The oligonucleotide comprising the barcode sequence may be further ligated to the oligonucleotide in the unmasked region to generate a barcoded oligonucleotide molecule.

In some cases, a primer extension or other amplification reaction may be used to synthesize an oligonucleotide on a substrate via a primer attached to the substrate. In such cases, a primer attached to the substrate may hybridize to a primer binding site of an oligonucleotide that also contains a template nucleotide sequence. The primer can then be extended by a primer extension reaction or other amplification reaction, and an oligonucleotide complementary to the template oligonucleotide can thereby be attached to the substrate.

In some embodiments, chemical ligation can be used to ligate two or more oligonucleotides. In some embodiments, chemical ligation involves the use of condensing reagents. In some embodiments, condensing reagents are utilized to activate a phosphate group. In some embodiments, condensing reagents may be one or more of 1-ethyl-3-(3-dimethylaminopropyl) carbodiimide (EDCI), cyanogen bromide, imidazole derivatives, and 1-hydroxybenzotriazole (HOAt). In some embodiments, functional group pairs selected from one or more of a nucleophilic group and an electrophilic group, or an alkyne and an azide group are used for chemical ligation. In some embodiments, chemical ligation of two or more oligonucleotides requires a template strand that is complementary to the oligonucleotides to be ligated (e.g., a splint). In some embodiments, the chemical ligation process is similar to oligonucleotide synthesis.

A splint is an oligonucleotide that, when hybridized to other polynucleotides, acts as a “splint” to position the polynucleotides next to one another so that they can be ligated together. In some embodiments, the splint is DNA or RNA. The splint can include a nucleotide sequence that is partially complimentary to nucleotide sequences from two or more different oligonucleotides. In some embodiments, the splint assists in ligating a “donor” oligonucleotide and an “acceptor” oligonucleotide. In general, an RNA ligase, a DNA ligase, or another other variety of ligase is used to ligate two nucleotide sequences together.

Splints have been described, for example, in US20150005200A1, the content of which is herein incorporated by reference in its entirety. A splint may be used for ligating two oligonucleotides. For example, the sequence of a splint may be configured to be in part complementary to at least a portion of the first oligonucleotides that are attached to the substrate and in part complementary to at least a portion of the second oligonucleotides. In one case, the splint can hybridize to the second oligo via its complementary sequence; once hybridized, the second oligonucleotide or oligonucleotide segment of the splint can then be attached to the first oligonucleotide attached to the substrate via any suitable attachment mechanism, such as, for example, a ligation reaction. The splint complementary to both the first and second oligonucleotides can then be then denatured (or removed) with further processing. The method of attaching the second oligonucleotides to the first oligonucleotides can then be optionally repeated to ligate a third, and/or a fourth, and/or more parts of the barcode onto the array with the aid of splint(s). In some embodiments, the splint is between 6 and 50 nucleotides in length, e.g., between 6 and 45, 6 and 40, 6 and 35, 6 and 30, 6 and 25, or 6 and 20 onucleotides in length. In some embodiments, the splint is between 15 and 50, 15 and 45, 15 and 40, 15 and 35, 15 and 30, 15 and 30, or 15 and 25 nucleotides in length.

In some embodiments, the method for providing an array described herein comprises (a) irradiating a substrate comprising an unmasked first region and a masked second region, whereby a photoresist in the first region is degraded to render oligonucleotide molecules in the first region available for hybridization and/or ligation, whereas oligonucleotide molecules in the second region are protected by the photoresist in the second region from hybridization and/or ligation; and (b) contacting oligonucleotide molecules in the first region with a first splint and a first oligonucleotide comprising a first barcode sequence. In some embodiments, the first splint hybridizes to the first oligonucleotide and the oligonucleotide molecules in the first region. In some embodiments, the first oligonucleotide is not ligated to oligonucleotide molecules in the second region. In some embodiments, the hybridization region between the first splint and the oligonucleotide molecules is at least 3, 4, 5, 6, 7, 8, 9, 10 bp or more than 10 bp. In some embodiments, the hybridization region between the first splint and the first oligonucleotide is at least 3, 4, 5, 6, 7, 8, 9, 10 bp or more than 10 bp.

In some embodiments, the oligonucleotide is ligated using the splint as template without gap filling prior to the ligation. In some embodiments, the oligonucleotide is ligated using the splint as template with gap filling prior to the ligation. In some embodiments, hybridization to the first splint brings the terminal nucleotides of the first oligonucleotide and the oligonucleotide molecules immediately next to each other, and the ligation does not require gap-filling. In some embodiments, hybridization to the first splint brings the terminal nucleotides of the first oligonucleotide and the oligonucleotide molecules next to each other and separated by one or more nucleotides, and the ligation is preceded by gap-filling. In some embodiments, the splint is removed after the ligation.

In some embodiments according to the method for providing an array described herein, the photoresist is a first photoresist. In some embodiments, the first oligonucleotide is ligated to the oligonucleotide molecules in the first region to generate first extended oligonucleotide molecules. In some embodiments, the method further comprises the following steps: (c) applying a second photoresist to the substrate, optionally wherein the second photoresist is applied after the first photoresist is removed from the substrate; (d) irradiating the substrate while the first region is masked and the second region is unmasked, whereby the first or second photoresist in the second region is degraded to render oligonucleotide molecules in the second region available for hybridization and/or ligation, whereas the first extended oligonucleotide molecules in the first region are protected by the second photoresist in the first region from hybridization and/or ligation; and (e) contacting oligonucleotide molecules in the second region with a second splint and a second oligonucleotide comprising a second barcode sequence. In some embodiments, the second splint hybridizes to the second oligonucleotide and the oligonucleotide molecules in the second region. In some embodiments, the second oligonucleotide is ligated to the oligonucleotide molecules in the second region to generate second extended oligonucleotide molecules. In some embodiments, the second oligonucleotide is not ligated to the first extended oligonucleotide molecules in the first region. In some embodiments according to the methods described in the section, steps (a)-(b) are part of a first cycle, steps (d)-(e) are part of a second cycle, and steps (a)-(e) are part of a first round, and wherein the method comprises one or more additional rounds. In some embodiments, steps (a)-(e) are part of a first round, the first and second oligonucleotides are Round 1 oligonucleotides, the first and second barcode sequences are Round 1 barcode sequences. In some embodiments, the method further comprises: a′) irradiating the substrate while the first region is unmasked and the second region is masked, whereby a photoresist in the first region is degraded to render the first extended oligonucleotide molecules in the first region available for hybridization and/or ligation, whereas the second extended oligonucleotide molecules in the second region are protected by the photoresist in the second region from hybridization and/or ligation; and (b′) attaching a first Round 2 oligonucleotide comprising a first Round 2 barcode sequence to the first extended oligonucleotide molecules in the first region via hybridization and/or ligation, wherein the second extended oligonucleotide molecules in the second region do not receive the first Round 2 barcode sequence. In some embodiments wherein the photoresist is a first photoresist, and the first Round 2 oligonucleotide is ligated to the first extended oligonucleotide molecules in the first region to generate first further extended oligonucleotide molecules, the method further comprises: (c′) applying a second photoresist to the substrate, optionally wherein the second photoresist is applied after the first photoresist is removed from the substrate; (d′) irradiating the substrate while the first region is masked and the second region is unmasked, whereby the first or second photoresist in the second region is degraded to render the second extended oligonucleotide molecules in the second region available for hybridization and/or ligation, whereas the first further extended oligonucleotide molecules in the first region are protected by the second photoresist in the first region from hybridization and/or ligation; and (e′) attaching a second Round 2 oligonucleotide comprising a second Round 2 barcode sequence to the second extended oligonucleotide molecules in the second region via hybridization and/or ligation, wherein the first further extended oligonucleotide molecules in the first region do not receive the second Round 2 barcode sequence. In some embodiments, the Round 1 barcode sequences are different from each other. In some embodiments, the Round 1 barcode sequences are different from the Round 2 barcode sequences.

In some aspects, provided herein is a method for construction of a hybridization complex or an array comprising nucleic acid molecules and complexes. FIG. 1B shows an exemplary oligonucleotide probe for capturing analytes or proxies thereof which may be generated using a method disclosed herein comprising four rounds of hybridization and ligation.

In some embodiments, the oligonucleotide probe can directly capture an analyte, such as mRNAs based on a poly(dT) capture domain on the oligonucleotide probe immobilized on an array. In some embodiments, the oligonucleotide probe is used for indirect analyte capture. For example, in fixed samples, such as FFPE, a probe pair can be used, and probe pairs can be target specific for each gene of the transcriptome. The probe pairs are delivered to a tissue section (which is itself on a spatial array) with a decrosslinking agent and a ligase, and the probe pairs are left to hybridize and ligate, thereby forming ligation products. The ligation products contain sequences in one or more overhangs of the probes, and the overhangs are not target specific and are complementary to capture domains on oligonucleotides immobilized on a spatial array, thus allowing the ligation product (which is a proxy for the analyte) to be captured on the array, processed, and subsequently analyzed (e.g., using a sequencing method).

In some embodiments, the oligonucleotide probe for capturing analytes or proxies thereof may be generated from an existing array with a ligation strategy. In some embodiments, an array containing a plurality of oligonucleotides (e.g., in situ synthesized oligonucleotides) can be modified to generate a variety of oligonucleotide probes. The oligonucleotides can include various domains such as, spatial barcodes, UMIs, functional domains (e.g., sequencing handle), cleavage domains, and/or ligation handles.

A “spatial barcode” may comprise a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate. A spatial barcode can be part of a capture probe on an array generated herein. A spatial barcode can also be a tag attached to an analyte (e.g., a nucleic acid molecule) or a combination of a tag in addition to an endogenous characteristic of the analyte (e.g., size of the analyte or end sequence(s)). A spatial barcode can be unique. In some embodiments where the spatial barcode is unique, the spatial barcode functions both as a spatial barcode and as a unique molecular identifier (UMI), associated with one particular capture probe. Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode is added to, for example, a fragment of a DNA or RNA sample before, during, and/or after sequencing of the sample. In some embodiments, a spatial barcode allows for identification and/or quantification of individual sequencing-reads. In some embodiments, a spatial barcode is a used as a fluorescent barcode for which fluorescently labeled oligonucleotide probes hybridize to the spatial barcode.

In some embodiments, a spatial array is generated after ligating capture domains (e.g., poly(T) or gene specific capture domains) to the oligonucleotides (e.g., generating capture oligonucleotides). The spatial array can be used with any of the spatial analysis methods described herein. For example, a biological sample can be provided to the generated spatial array. In some embodiments, the biological sample is permeabilized. In some embodiments, the biological sample is permeabilized under conditions sufficient to allow one or more analytes or proxies thereof in the biological sample to interact with the capture probes of the spatial array. After capture of analytes or proxies thereof from the biological sample, the analytes or proxies thereof can be analyzed (e.g., reverse transcribed or extended, amplified, and sequenced) by any of the variety of methods described herein.

For example, FIG. 1B shows the sequential hybridization/ligation of various domains to generate an oligonucleotide probe for capturing analytes or proxies thereof, by a photo-hybridization/ligation method described herein.

As illustrated in FIG. 1B, an oligonucleotide is immobilized on a substrate (e.g., an array) and may comprise a functional sequence such as a primer sequence. In some embodiments, the primer sequence is a sequencing handle that comprises a primer binding site for subsequent processing. The primer sequence can generally be selected for compatibility with any of a variety of different sequencing systems, e.g., 454 Sequencing, Ion Torrent Proton or PGM, Illumina X₁₀, PacBio, Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Roche 454 sequencing, Ion Torrent Proton or PGM sequencing, Illumina X₁₀ sequencing, PacBio SMRT sequencing, and Oxford Nanopore sequencing. Further, in some embodiments, functional sequences can be selected for compatibility with other sequencing systems, including non-commercialized sequencing systems.

Following a first cycle of photo-hybridization/ligation, an oligonucleotide comprising a part of a barcode (e.g., BC-A) is attached to the oligonucleotide molecule comprising the primer. In some embodiments, the barcode part can be common to all of the oligonucleotide molecules in a given feature. In some embodiments, the barcode part can be different for oligonucleotide molecules in different features. In some embodiments, a splint with a sequence complementary to a portion of the primer of the immobilized oligonucleotide and an additional sequence complementary to a portion of the oligonucleotide comprising BC-A facilitates the ligation of the immobilized oligonucleotide and the oligonucleotide comprising BC-A. In some embodiments, the splint for attaching part BC-A of various sequences to different features is common among the cycles of the same round.

In FIG. 1B, another cycle, such as a second cycle, of photo-hybridization/ligation involves the addition of another oligonucleotide comprising a part of a barcode (e.g., BC-B) to the immobilized oligonucleotide molecule comprising the primer and BC-A. In some embodiments, a splint with a sequence complementary to a portion of the immobilized oligonucleotide comprising BC-A and an additional sequence complementary to a portion of the oligonucleotide comprising BC-B facilitates the ligation of the oligonucleotide comprising BC-B and the immobilized oligonucleotide comprising BC-A. In some embodiments, the splint for attaching part BC-B of various sequences to different features is common among the cycles of the same round.

FIG. 1B further illustrates a third cycle of photo-hybridization/ligation, which involves the addition of another oligonucleotide comprising a part of a barcode (e.g., BC-C), added to the immobilized oligonucleotide molecule comprising the primer, BC-A, and BC-B. In some embodiments, a splint with a sequence complementary to a portion of the immobilized oligonucleotide molecule comprising BC-B and an additional sequence complementary to a portion of the oligonucleotide comprising BC-C facilitates the ligation of the immobilized oligonucleotide molecule comprising BC-B and the oligonucleotide comprising BC-C. In some embodiments, the splint for attaching part BC-C of various sequences to different features is common among the cycles of the same round.

A fourth cycle of photo-hybridization/ligation may be performed, which involves the addition of another oligonucleotide comprising a part of a barcode (e.g., BC-D), added to the immobilized oligonucleotide molecule comprising the primer, BC-A, BC-B, and BC-C. In some embodiments, a splint with a sequence complementary to a portion of the immobilized oligonucleotide molecule comprising BC-C and an additional sequence complementary to a portion of the oligonucleotide comprising BC-D facilitates the ligation. In some embodiments, the splint for attaching part BC-D of various sequences to different features is common among the cycles of the same round. In some embodiments, as shown in FIG. 1B, an oligonucleotide comprising BC-D further comprises a UMI and a capture domain.

In some embodiments, the splint comprises a sequence that is complementary to an oligonucleotide (e.g., an immobilized oligonucleotide), or a portion thereof, and a sequence that is complementary to an oligonucleotide containing a barcode, or a portion thereof. In some embodiments, the splint comprises a sequence that is perfectly complementary (e.g., is 100% complementary) to an oligonucleotide (e.g., an immobilized oligonucleotide), or a portion thereof, and/or a sequence that is perfectly complementary to an oligonucleotide containing a barcode, or a portion thereof. In some embodiments, the splint comprises a sequence that is not perfectly complementary (e.g., is not 100% complementary) to an oligonucleotide (e.g., an immobilized oligonucleotide), or a portion thereof, and/or a sequence that is not perfectly complementary to an oligonucleotide containing a barcode, or a portion thereof. In some embodiments, the splint comprises a sequence that is at least 70%, 75%, 80%, 85%, 90%, 95%, 96%, 97%, 98%, or 99% complementary to an oligonucleotide (e.g., an immobilized oligonucleotide), or a portion thereof, and/or a sequence that is complementary to an oligonucleotide containing a barcode, or a portion thereof. In some embodiments, the splint comprises a sequence that is perfectly complementary (e.g., is 100% complementary) to an oligonucleotide (e.g., an immobilized oligonucleotide), or a portion thereof, but is not perfectly complementary to a sequence that is complementary to an oligonucleotide containing a barcode, or a portion thereof. In some embodiments, the splint comprises a sequence that is not perfectly complementary (e.g., is not 100% complementary) to an oligonucleotide (e.g., an immobilized oligonucleotide), or a portion thereof, but is perfectly complementary to a sequence that is complementary to an oligonucleotide containing a barcode, or a portion thereof. So long as the splint is capable of hybridizing to an oligonucleotide (e.g., an immobilized oligonucleotide), or a portion thereof, and to a sequence that is complementary to an oligonucleotide containing a barcode, or a portion thereof, the splint need not have a sequence that is perfectly complementary to either the oligonucleotide (e.g., the immobilized oligonucleotide) or to the an oligonucleotide containing a barcode.

In some embodiments, oligonucleotides that are exposed and do not receive a ligated oligonucleotide could receive the incorrect barcode during the next cycle or round. In order to prevent generating the wrong barcode at the wrong spot, unligated oligonucleotides may be rendered unavailable for hybridization and/or ligation, e.g., the unligated oligonucleotides can be capped and/or removed. In some embodiments, the oligonucleotides are modified at the 3′. Non-limiting examples of 3′ modifications include dideoxy C-3′ (3′-ddC), 3′ inverted dT, 3′ C3 spacer, 3′Amino, and 3′ phosphorylation.

In some embodiments according to any of the methods described herein, the method further comprises blocking the 3′ or 5′ termini of barcoded oligonucleotide molecules. In some embodiments, the blocking comprises adding a 3′ dideoxy or a non-ligating 3′ phosphoramidite to the barcoded oligonucleotide molecules. In some embodiments, the blocking comprises adding a 3′ dideoxy or a non-ligating 3′ phosphoramidite to the barcoded oligonucleotide molecules. In some embodiments, the blocking comprises adding a 3′ dideoxy or a non-ligating 3′ phosphoramidite to the unligated oligonucleotide molecules. In some embodiments, the addition is catalyzed by a terminal transferase. In some embodiments, the terminal transferase is TdT. The blocking may be removed after the blocking reaction is completed. In some embodiments, the blocking is removed using an internal digestion of the barcoded oligonucleotide molecules after ligation is completed.

### IV. LIGHT-CONTROLLED SURFACE PATTERNING IN SITU USING PHOTOMASKS

In some aspects, provided herein is a method of patterning a surface in situ using one or more photomasks for producing an array on the surface, for example, by spatially-selective light-activated hybridization/ligation generating unique DNA sequences in unique spatial positions in the array. In any of the embodiments herein, the method disclosed herein can comprise a photo-hybridization ligation that is performed using exemplary methods and reagents described in US 2022/0228201 A1, US 2022/0228210 A1, and US 2022/0314187 A1, each of which is incorporated here by reference in its entirety for all purposes. For instance, some oligonucleotides on a substrate can be blocked (e.g., via photo-cleavable polymers, photo-cleavable moieties, or a photoresist) from hybridization and ligation while other oligonucleotides on the substrate can undergo hybridization and ligation to attach a nucleic acid barcode or part thereof. The deblocking can comprise: photo-cleaving a polymer that blocks an oligonucleotide in a prior cycle from hybridization and ligation; removing a photo-cleavable moiety of an oligonucleotide that blocks the oligonucleotide in a prior cycle from hybridization and ligation; or removing a photoresist that blocks the oligonucleotide in a prior cycle from hybridization and ligation.

In some embodiments, the method comprises assembling barcode sequences on immobilized oligonucleotides, e.g., based on hybridization and/or ligation, on a slide or wafer surface. In some embodiments, the in situ method comprises photolithography using one or more photomasks to enable barcodes to be generated combinatorially, for example, in as few as three rounds of assembly. In some embodiments, exposure of the photoresist to irradiation may render the exposed regions dissolvable by a developer. In some embodiments, the photoresist in the unmasked region of the substrate is dissolved by a developer and removed. The developer may be organic or aqueous based. A non-limiting example of an aqueous base developer include tetramethylammonium hydroxide aqueous solution.

In some embodiments, the method comprises assembling barcode sequences on immobilized oligonucleotides, e.g., based on hybridization and/or ligation, on a slide surface. In some embodiments, the in situ method uses photoresist and photolithography to enable barcodes to be generated selectively on a discrete location on a slide surface. Hybridization and/or ligation of barcodes can be controlled, for example, using a contact photolithography process. For example, ligation can be achieved by exposing oligonucleotides for ligation upon degradation of a photoresist by irradiating a substrate through a photomask.

In some embodiments, the method comprises irradiating a substrate covered with a photoresist through one or more photomasks. In some embodiments, the irradiation is selective, for example, where one or more photomasks can be used such that only one or more specific regions of the array are exposed to irradiation stimuli (e.g., exposure to light such as UV, and/or exposure to heat induced by laser).

In some embodiments, the oligonucleotide molecules on the substrate comprise one or more common sequences. In some embodiments, the one or more common sequences comprise a common primer sequence. The common primer sequence can be of about 5, about 6, about 7, about 8, about 9, about 10, about 15, about 20, about 25, about 30, about 35, about 40, about 45, about 50, or about 60 nucleotides in length. In some embodiments, the common primer sequence is between about 10 and about 35 nucleotides in length.

In some embodiments, the array comprises an arrangement of a plurality of features, e.g., each comprising one or more molecules such as a nucleic acid molecule (e.g., a DNA oligo). In some embodiments, the array comprises different oligonucleotides in different features. In some embodiments, oligonucleotide molecules on the substrate are immobilized in a plurality of features. Nucleotides immobilized on the substrate may be of different orientations. For example, in some embodiments, the 3′ terminal nucleotides of immobilized oligonucleotide molecules are distal to the substrate. In some embodiments, the 5′ terminal nucleotides of immobilized oligonucleotide molecules are distal to the substrate.

The oligonucleotide molecules on the substrate prior to the irradiating step may have a variety of properties, which include but are not limited to, length, orientation, structure, and modifications. The oligonucleotide molecules on the substrate prior to the irradiating step can be of about 4, about 5, about 6, about 7, about 8, about 9, about 10, about 15, about 20, about 25, about about 35, about 40, about 45, about 50, about 55, about 60, about 70, about 80, about 90, or about 100 nucleotides in length. In some embodiments, oligonucleotide molecules on the substrate prior to the irradiating step are between about 5 and about 50 nucleotides in length. The oligonucleotide molecules on the substrate may comprise functional groups. In some embodiments, the functional groups are amino or hydroxyl groups. The functional groups can be protected or unprotected. In some embodiments, the functional groups are not protected, e.g., by a photo-sensitive group, moiety, or molecule prior to the irradiating step. In some embodiments, the functional groups are 3′ hydroxyl groups of nucleotides.

In some embodiments, the method provided herein comprises using a series of photomasks, oligonucleotides in desired regions of the lawn may be iteratively deprotected via exposure to light and removal of the photoresist.

In some embodiments, the method further comprises attaching a round 1 barcode to one or more exposed oligonucleotides, for example, by attaching an oligo cassette with a complementary region (e.g., complementary to a splint) and a barcode region (e.g. a first barcode sequence). In some embodiments, the attachment may be performed by placing the substrate in a chamber or vessel (e.g., within which oligonucleotides such as those comprising barcode sequences can be delivered and ligated to nucleic acid molecules on the substrate). In some embodiments, the chamber or vessel is a flow cell or a device comprising microfluidic channels. In some embodiments, the method comprises flowing in the round 1 barcode (e.g., an oligo cassette) to be attached to the common oligonucleotide. The process can be repeated N cycles (each cycle for one or more features on an array) for round 1 until all desired features have been exposed (e.g., due to exposure of the photoresist covering the features to light) and the common oligonucleotides in the features have received the round 1 barcode which may be the same or different for molecules in any two given features. The round 1 barcode molecules (e.g. a first oligonucleotide comprising a first barcode sequence) can be ligated to the common oligonucleotides. The process can be repeated M rounds to achieve a desired barcode diversity, for example, by attaching a round 2 barcode (which may be the same or different for molecules in any two given features), a round 3 barcode (which may be the same or different for molecules in any two given features), . . . , and a round m barcode (which may be the same or different for molecules in any two given features) to each of the growing oligonucleotides in the features. In some embodiments, each round comprises a plurality of cycles (each cycle for one or more features on an array) of photoresist exposure to light and oligonucleotide attachment until all desired features have been exposed once and the molecules in the features have received the barcode(s) (which may be the same or different for molecules in any two given features) for that round. In some embodiments, the method further comprises attaching a capture sequence to the barcoded oligonucleotides, for example, by hybridization and/or ligation.

In some aspects, a method disclosed herein provides one or more advantages as compared to other arraying methods. For example, pre-synthesized barcodes can eliminate concern over barcode fidelity in base-by-base in situ approaches. In addition, compared to base-by-base method, a method disclosed herein can reduce time and increase total yield. For example, only three or four rounds may be required as compared to 12-16 rounds in a typical base-by-base in situ arraying method. In one aspect, the method disclosed herein does not involve 5′ to 3′ base-by-base synthesis of a polynucleotide in situ on a substrate. In one aspect, there is minimal need for manipulating photomasks in the irradiation step. In another aspect, there is no need for decoding as all barcodes are synthesized in defined locations on an array. In some aspects, feature scaling can readily be increased or decreased by changing photomasks and corresponding barcode diversity. In other aspects, a method disclosed herein is performed on a transparent substrate. Since a method disclosed herein does not depend on the use of microspheres (e.g., barcoded beads) to generate an oligonucleotide array, optical distortion or aberrations caused by microspheres (which may not be transparent) during imaging of the array and/or a sample (e.g., a tissue section) on the array can be avoided.

In some aspects, provided herein is a method of producing an array of polynucleotides. In some embodiments, an array comprises an arrangement of a plurality of features, e.g., each comprising one or more molecules such as a nucleic acid molecule (e.g., a DNA oligo), and the arrangement is either irregular or forms a regular pattern. The features and/or molecules on an array may be distributed randomly or in an ordered fashion, e.g. in spots that are arranged in rows and columns. Individual features in the array differ from one another based on their relative spatial locations. In some embodiments, the features and/or molecules are collectively positioned on a substrate.

In some embodiments, polynucleotides of the same or different nucleic acid sequences are immobilized on the substrate in a pattern prior to the irradiation. In some embodiments, the pattern comprises rows and/or columns. In some embodiments, the pattern comprises regular and/or irregular shapes (e.g., polygons).

In some embodiments, the method comprises irradiating an array with light. In some embodiments, the irradiation is selective, for example, where one or more photomasks can be used such that only one or more specific regions of the array are exposed to stimuli (e.g., exposure to light such as UV, and/or exposure to heat induced by laser).

In some embodiments, the oligonucleotide molecules are prevented by the photoresist from hybridization to a nucleic acid such as a splint. In some embodiments, the oligonucleotide molecules are prevented by the photoresist from ligation to a nucleic acid. For example, the photoresist may inhibit or block the 3′ or 5′ end of an oligonucleotide molecule from chemical or enzymatic ligation, e.g., even when a splint may hybridize to the oligonucleotide molecule in order to bring a ligation partner in proximity to the 3′ or 5′ end of the oligonucleotide molecule. In some embodiments, the 3′ or 5′ end of the oligonucleotide molecule or a hybridization/ligation product thereof is capped.

In some embodiments, the irradiation results in degradation of the photoresist such that the inhibition or blocking of hybridization and/or ligation to an oligonucleotide molecule in an exposed (e.g., unmasked) region is reduced or eliminated, whereas hybridization and/or ligation to an oligonucleotide molecule in an unexposed (e.g., masked) region remains inhibited or blocked by a photoresist which may be the same or different from the degraded photoresist.

In some embodiments, the method further comprises attaching a first barcode molecule (e.g. the first oligonucleotide) comprising a first barcode sequence to an oligonucleotide molecule in an exposed (e.g., unmasked) region via hybridization and/or ligation. In some embodiments, one end of the first barcode molecule and one end of the oligonucleotide molecule may be directly ligated, e.g., using a ligase having a single-stranded DNA/RNA ligase activity such as a CircLigase™. The attachment may comprise hybridizing the first barcode molecule and the oligonucleotide molecule to a splint, wherein one end of the first barcode molecule and one end of the oligonucleotide molecule are in proximity to each other. For example, the 3′ end of the first barcode molecule and the 5′ end of the oligonucleotide molecule may hybridize to a splint. Alternatively, the 5′ end of the first barcode molecule and the 3′ end of the oligonucleotide molecule are in proximity to each other. In some embodiments, proximity ligation is used to ligate a nick, with or without a gap-filling step that involves incorporation of one or more nucleic acids by a polymerase, based on the nucleic acid sequence of the splint which serves as a template.

In some embodiments, a first polynucleotide (e.g., an oligo) is deposited in a region A of a substrate and a second polynucleotide (e.g., an oligo) is deposited in a region B. Regions A are exposed to light while regions B are masked by a photomask. A photomask can be selected and/or adjusted to allow any suitable number and/or combination of regions on the substrate to be exposed to light or masked. Thus, the exposed region(s) and masked region(s) can be in any suitable pattern, which can be predetermined and/or adjusted as needed during the arraying process. In addition, a mirror, mirror array, a lens, a moving stage, and/or a photomask can be used to direct the light to or away from the region(s) of interest. In some embodiments, the first polynucleotide and the second polynucleotide can comprise the same sequence or different sequences. For example, first polynucleotides in region A and second polynucleotides in region B may form a lawn of universal oligo molecules on the substrate. The oligonucleotides may be attached to the substrate at their 5′ ends or 3′ ends. The first and second polynucleotides can be embedded in a first and a second photoresist, respectively. The first and second photoresist can be the same or different. In some embodiments, first polynucleotides in region A and second polynucleotides in region B are embedded in the same photoresist layer. Once regions A are exposed to light to deprotect the first polynucleotide while the second polynucleotide in regions B remain protected, a first barcode can be attached to the first polynucleotide. In some embodiments, a hybridization complex is formed between the first polynucleotide, a splint, and a polynucleotide comprising a first barcode (e.g., a round 1 barcode 1A). The polynucleotide comprising the first barcode comprise at least a first barcode sequence and a hybridization region that hybridizes to the splint which is a first splint, and may further comprise a hybridization region that hybridizes to a round 2 splint (e.g., for attaching a round 2 barcode after the round 1 barcode 1A). The first splint comprises at least a hybridization region that hybridizes to the first polynucleotide and a hybridization region that hybridizes to the polynucleotide comprising the first barcode. Optionally, the polynucleotide comprising the first barcode may be ligated to the first polynucleotide, with or without gap filling using the first splint as a template. As a result, provided in some embodiments is an array comprising the first and second polynucleotides, wherein the first polynucleotide is barcoded with the first barcode and the second polynucleotide is not, and neither of the barcoded first polynucleotide nor the second polynucleotide comprises a photo-cleavable moiety.

In some embodiments, the polynucleotide comprising the first barcode may comprise no photo-cleavable moiety that blocks hybridization and/or ligation. In these examples, the array may be exposed to light to degrade photoresist that protects the second polynucleotide, and a second barcode can be attached to the second polynucleotide. In some embodiments, a hybridization complex is formed between the second polynucleotide, a second splint, and a polynucleotide comprising a second barcode (e.g., a round 1 barcode 1B). The polynucleotide comprising the second barcode comprises at least a second barcode sequence and a hybridization region that hybridizes to the second splint, and may further comprise a hybridization region that hybridizes to a round 2 splint (e.g., for attaching a round 2 barcode after the round 1 barcode 1B). The second splint comprises at least a hybridization region that hybridizes to the second polynucleotide and a hybridization region that hybridizes to the polynucleotide comprising the second barcode. While the polynucleotide comprising the first barcode may be available for hybridization and/or ligation, the second barcode may be specifically attached to the second polynucleotide but not to the first polynucleotide barcoded with the first barcode. For example, the sequence of the second splint may be selected such that it specifically hybridizes to the second polynucleotide but not to the polynucleotide comprising the first barcode. In these examples, both the first barcode (e.g., barcode 1A) and the second barcode (e.g., barcode 1B) are round 1 barcodes. Optionally, the polynucleotides comprising the first/second barcodes may be ligated to the first/second polynucleotides, respectively, with or without gap filling using the first/second splints as templates. As a result, provided in some embodiments is an array comprising the first and second polynucleotides barcoded with the first barcode and the second barcode, respectively, wherein neither of the barcoded polynucleotides comprises a photo-cleavable moiety.

In some examples, polynucleotides in regions A and/or polynucleotides in regions B may undergo one or more additional rounds of barcoding. For example, after the round 1 barcoding, regions A may contain polynucleotides P1 and P3 each barcoded with round 1 barcode 1A (i.e., polynucleotides 1A-P1 and 1A-P3) and regions B may contain polynucleotides P2 and P4 each barcoded with round 1 barcode 1B (i.e., polynucleotides 1B-P2 and 1B-P4). All of polynucleotides 1A-P1, 1A-P3, 1B-P2, and 1B-P4 may be embedded in a photoresist. With light exposure and photomasking, any one or more of polynucleotides 1A-P1 and 1A-P3 (in regions A) and 1B-P2 and 1B-P4 (in regions B) may undergo a second round of barcoding.

For instance, a round 2 barcode 2A may be attached to any one of polynucleotides 1A-P1, 1A-P3, 1B-P2, and 1B-P4. In some embodiments, a round 2 barcode 2A may be attached to any two of polynucleotides 1A-P1, 1A-P3, 1B-P2, and 1B-P4. In some embodiments, a round 2 barcode 2A may be attached to any three of polynucleotides 1A-P1, 1A-P3, 1B-P2, and 1B-P4. In some embodiments, a round 2 barcode 2A may be attached to all of polynucleotides 1A-P1, 1A-P3, 1B-P2, and 1B-P4.

In other examples, different round 2 barcodes 2A and 2B may be used. In some embodiments, barcode 2A is attached to polynucleotides 1A-P1 and 1A-P3 (in regions A) while barcode 2B is attached to polynucleotides 1B-P2 and 1B-P4 (in regions B). For higher order rounds, for example, round m (m being an integer of 2 or greater), the regions A polynucleotides may receive barcode mA while the regions B polynucleotides receive barcode mB. Barcodes mA and mB may be the same or different in sequence. Thus, for each round, the regions A polynucleotides (e.g., P1 and P3) and the regions B polynucleotides (e.g., P2 and P4) may have no crossover, generating barcoded polynucleotides mA- . . . -1A-P1 and mA- . . . -1A-P3 (in regions A) and mB- . . . -1B-P2 and mB- . . . -1B-P4 (in regions B).

Alternatively, the regions A polynucleotides (e.g., P1 and P3) and the regions B polynucleotides (e.g., P2 and P4) may have crossover. For example, barcode 2A is attached to polynucleotides 1A-P1 (in regions A) and 1B-P2 (in regions B) while barcode 2B is attached to polynucleotides 1A-P3 (in regions A) and 1B-P4 (in regions B). For example, barcoded polynucleotides 2A-1A-P1 and 2B-1A-P3 (in regions A) and 2A-1B-P2 and 2B-1B-P4 (in regions B) may be generated. For round m (m being an integer of 2 or greater), one or more of the regions A polynucleotides and/or one or more of the regions B polynucleotides may receive barcode mA, while one or more of the regions A polynucleotides and/or one or more of the regions B polynucleotides barcode mB. Barcodes mA and mB may be the same or different in sequence.

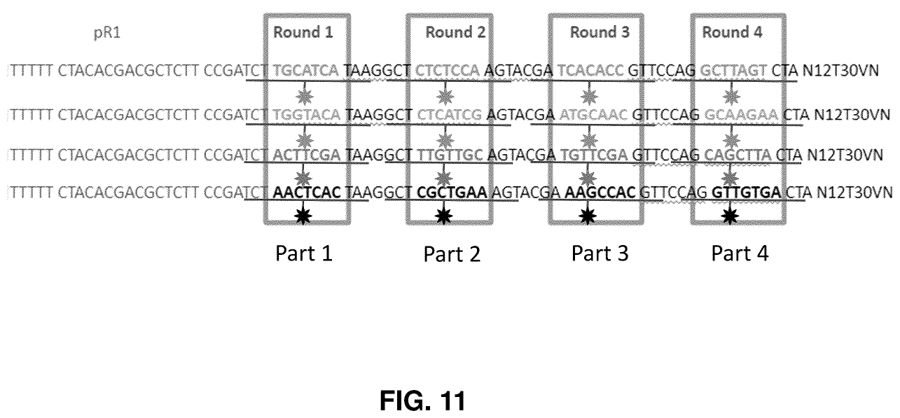

In some examples, round m (m being an integer of 2 or greater) barcodes mA, mB, and mC may be attached to any polynucleotides barcoded in the previous round (i.e., round m-/), and mA, mB, and mC may be the same or different. In other examples, round m (m being an integer of 2 or greater) barcodes mA, mB, mC, and mD may be attached to any polynucleotides barcoded in the previous round (i.e., round m−1), and mA, mB, mC, and mD may be the same or different. Exemplary barcodes synthesized by 4 rounds of ligation are shown in FIG. 11. The barcoded polynucleotides shown in FIG. 11 are set forth in SEQ ID NOS:1-4. In addition, each of the four barcoded polynucleotides include an UMI+ Capture sequence, which is set forth in SEQ ID NO:5, and is depicted as “N12T3OVN” in FIG. 11, consisting of the sequence NNNNNNNNNNNNTTTTTTTTTTTTTTTTTTTTTTTTTTTTTTVN, where N is any nucleotide and V is A, C, or G.

In any of the preceding embodiments, the barcoding rounds can be repeated m times to achieve a desired barcode diversity, m being an integer of 2 or greater. In some embodiments, m is 3, 4, 5, 6, 7, 8, 9, or 10, or greater than 10. In any of the preceding embodiments, each of the m barcoding rounds may comprise n cycles (each cycle for molecules in one or more features), wherein integer n is 2 or greater and independent of m. In some embodiments, n is 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater than 50.

In some embodiments, the oligonucleotide molecules on the substrate are immobilized in a plurality of features (e.g. regions). In some embodiments, the feature(s) may be no more than 0.5 micron, no more than 1 micron, no more than 3 microns, no more than 5 microns, no more than 10 microns, or no more than 15 microns, no more than 20 microns, no more than 25 microns, no more than 30 microns, or no more than 35 microns, no more than 40 microns, no more than 45 microns, or no more than 50 microns in diameter. In some embodiments, the features on the substrate are below 10 microns in diameter (e.g., single cell scale resolution) and provide high throughput readout (e.g., by sequencing) for analyzing a sample, such as a tissue sample.

FIG. 1A provides a non-limiting example, where the substrate comprises a surface for nucleic acids to be deposited on and can be in the form of a slide, such as a glass slide. In some examples, the substrate is transparent. A lawn of polynucleotides (e.g. oligonucleotide lawn) without photo-cleavable moieties, such as photo-caged oligonucleotides, may be deposited on the substrate and immobilized. A photoresist may be coated onto the substrate and cover the polynucleotides. One or more regions (e.g., regions A) on the substrate are exposed to light in order to degrade the photoresist, rendering the polynucleotides in the one or more regions available for hybridization and/or ligation, while one or more other regions (e.g., regions B) on the substrate are masked or not exposed to light. Patterned exposure of polynucleotides on the underlying substrate is provided, and a round 1 barcode (such as barcode 1A) may be attached to the exposed polynucleotides via hybridization and/or ligation. For example, an oligo may be used to hybridize to an exposed polynucleotide and a polynucleotide comprising the round 1 barcode. The oligo may comprise a splint that facilitates proximity ligation of one end of the exposed polynucleotide and one end of the polynucleotide comprising the round 1 barcode, thus attaching the barcode to the exposed polynucleotide. The proximity ligation may occur immediately following hybridization, in a subsequent step of the same cycle, or in a subsequent cycle (for example, while molecules comprising barcode 1B are ligated to polynucleotides in regions B). Thus, the one or more regions (e.g., regions A) on the substrate contain polynucleotides barcoded with the round 1 barcode (such as barcode 1A), while the one or more other regions (e.g., regions B) on the substrate do not contain polynucleotides barcoded with the round 1 barcode. In some embodiments, the photoresist covering polynucleotides in the one or more other regions (e.g., regions B) is not removed prior to the next cycle where a photoresist is applied to cover polynucleotides in the one or more regions (e.g., regions A). In some embodiments, the photoresist covering polynucleotides in the one or more other regions (e.g., regions B) is removed prior to the next cycle. In some embodiments, the photoresist is removed from the substrate (e.g., all regions on the substrate) prior to the next cycle, and a new layer of a photoresist composition (which may be the same or different from the removed photoresist composition) is applied to the substrate, e.g., to cover both regions A and regions B. In the next cycle, the polynucleotides barcoded with 1A in regions A are masked while regions B are exposed to light in order to degrade the photoresist and expose the polynucleotides in regions B. Another round 1 barcode (barcode 1B) may be attached to the exposed polynucleotides in regions B via hybridization and/or ligation, for example, as described above for attaching barcode 1A. FIG. 1B shows the sequential hybridization/ligation of various domains to generate an oligonucleotide probe for capturing analytes or proxies thereof, by a photo-hybridization/ligation method described herein.

In some embodiments, as shown in FIG. 2, a method disclosed herein comprises M rounds, where each round comprises N cycles (an exemplary cycle is shown in FIG. 1) to achieve a desired barcode diversity up to N^{M}, wherein M and N are integers independent of each other and are at least 2.

In some embodiments, the number of photomasks may be minimized by rotating and/or translating one of more photomasks in between rounds and/or cycles, and/or by using a set of photomasks comprising openings of different sizes.

In some aspects, disclosed herein is a method for providing an array with photomasks, comprising: irradiating a plurality of regions on a substrate through a first photomask comprising openings that correspond to all or a subset of the plurality of regions, wherein a first oligonucleotide of at least four nucleotides in length is attached to oligonucleotide molecules in the plurality of regions to generate extended oligonucleotide molecules; (b) irradiating the plurality of regions through the first photomask rotated relative to the substrate, or through a second photomask comprising openings that correspond to all or a subset of the plurality of regions, wherein a second oligonucleotide of at least four nucleotides in length is attached to the extended oligonucleotide molecules in the plurality of regions to generate further extended oligonucleotide molecules. In some aspects, the plurality of regions may be arranged in rows and columns on the substrate, and the first photomask and/or the second photomask comprises openings that correspond to one or more of the rows or one or more of the columns. In some aspects, the plurality of regions may be arranged in hexagonal arrays.

In some embodiments, provided herein are methods of generating arrays comprising square regions of unique barcodes with minimal photomask schemes. In an non-limiting example as illustrated in FIG. 3A, the first round of barcode array synthesis may comprise using a first photomask comprising square openings to generate a plurality of regions on a substrate. In some embodiments, the square openings correspond to all of the plurality of regions. In some embodiments, the square openings correspond to a subset of the plurality of regions, such as a column of four square regions as shown in FIG. 3A, and the first photomask is translated horizontally to irradiate the rest of the plurality of regions in subsequent cycles of the first round. 4 unique first parts of barcode sequences may be further ligated onto different square regions of the substrate in a column-wise manner. In some embodiments, the photomask used in different rounds may be the same between the first round and the second round. In some embodiments, the photomask may be rotated between the first round and the second round. As illustrated in FIG. 3B, during the second round where the second part of the barcode sequence is ligated onto the first part, the photomask used in the first round is rotated 90° and translated vertically to incorporate 4 unique second parts of the column onto different regions of the substrate in a row-wise manner. After the first two rounds, there is generated a substrate with 16 unique 2-part barcodes on 16 square regions. In some embodiments, a different photomask may be used to further ligate a third part of the barcode sequence. For example, FIG. 3C shows that by using a second mask comprising a plurality of smaller square openings (an opening the size of 1 grid in a 5×5 matrix within one of the 16 square regions generated in FIGS. 3A and 3B) in the non-limiting example in FIG. 3C), a finer pattern of barcode sequences may be generated within each square regions shown in FIGS. 3A and 3B by further ligating unique third parts of barcode sequences to the 2-part barcodes generated in FIG. 3B. In some embodiments, the spacing of the smaller square openings on the second photomask may be the same as the spacing of the square openings on the first photomask.

In some embodiments, provided herein are methods for generating an array comprising hexagonal regions of unique barcodes with minimal photomask schemes. In some non-limiting embodiments as illustrated in FIG. 4A, the first round of barcode array synthesis may comprise using a first photomask comprising one or more columns of hexagonal openings to generate a plurality of regions on a substrate, such as the columns in FIG. 4A comprising 8 hexagons. The first mask may be translated to cover the entire substrate and ligate unique first parts of barcodes onto the substrate. In the second round as illustrated in FIG. 4B, a second photomask with a plurality of hexagonal openings is used to ligate unique second parts of barcodes onto the first part in a row-wise manner, thereby generating 64 (8×8) unique 2-part barcodes. In some embodiments, a third photomask may be used to further ligate a third part of the barcode sequence to the hexagonal array. For example, FIG. 4C shows that by using a third mask comprising a plurality of smaller hexagonal openings, a finer pattern of barcode sequences may be generated within each hexagonal regions shown in FIGS. 4A and 4B by further ligating unique third parts of barcode sequences to the 2-part barcodes generated in FIG. 4B. In some embodiments, the spacing of the smaller hexagonal openings on the second photomask may be the same as the spacing of the hexagonal openings on the first and the second photomasks.

FIGS. 8A-8C show another example of using three photomasks in “coarse-fine” patterning to generate a hexagonal array. FIG. 8A shows in Round 1, a first photomask comprising periodic “fine” openings (one “fine” opening corresponding to a hexagonal region per 4×4 matrix of 16 hexagonal regions) can be used in 16 cycles to incorporate first barcode sequences into oligonucleotide molecules. FIG. 8B shows in Round 2, a second photomask comprising a “coarse” opening can be used in 17 cycles to incorporate second barcode sequences into oligonucleotide molecules. The “coarse” opening can be moved between cycles to correspond to one of the 17 rows each comprising 4 rows of hexagonal regions. FIG. 8C shows in Round 3, a third photomask comprising a “coarse” opening can be used in 15 cycles to incorporate third barcode sequences into oligonucleotide molecules. The “coarse” opening can be moved between cycles to correspond to one of the 15 columns, each column comprising 4 columns of hexagonal regions. Rounds 1-3 can be performed in any suitable order. For example, a first barcoding round can be a “coarse” round (e.g., 17×1) to barcode oligonucleotide molecules in each of the 17 rows on the substrate, followed by a second “coarse” round (e.g., 1×15) to barcode oligonucleotide molecules in each of the 15 columns on the substrate, and then followed by a “fine” round (e.g., 4×4) to barcode oligonucleotide molecules in each of the 16 hexagonal regions of the repeating 4×4 matrix. In another example, a first “coarse” barcoding round (e.g., either 1×15 or 17×1) can be followed by a “fine” round (e.g., 4×4), which is then followed by a second “coarse” round (e.g., 17×1 or 1×15).

In another aspect, disclosed herein is a method for providing an array with photomasks, comprising: (a) irradiating a plurality of regions arranged in columns and rows on a substrate through a first photomask comprising periodic openings that repeat every X₁ columns and every Y₁ rows, wherein each opening corresponds to a region within a X₁×Y₁ matrix on the substrate, and wherein a first oligonucleotide of at least four nucleotides in length is attached to oligonucleotide molecules in the plurality of regions to generate extended oligonucleotide molecules; and (b) irradiating the plurality of regions through a second photomask comprising periodic openings that repeat every X₂ columns and every Y₂ rows, wherein each opening corresponds to a region within a X₂×Y₂ matrix on the substrate, and wherein a second oligonucleotide of at least four nucleotides in length is attached to the oligonucleotide molecules in the plurality of regions to generate extended oligonucleotide molecules, thereby providing on the substrate an array comprising oligonucleotide molecules.

In another embodiment, there is provided a method of generating an array with minimal photomask schemes wherein regions on the array are arranged in columns and rows. In some embodiments, the method comprises use of photomasks comprising periodic openings. In some embodiments, the periodic openings on a first photomask repeat every X₁ columns and every Y₁ rows, wherein each opening corresponds to a region within a X₁×Y₁ matrix on the substrate. In some embodiments, the periodic openings on a second photomask repeat every X₂ columns and every Y₂ rows, wherein each opening corresponds to a region within a X₂×Y₂ matrix on the substrate. Similarly, in some embodiments, the periodic openings on a Nth photomask repeat every Xₙ columns and every Yₙ rows, wherein each opening corresponds to a region within a Xₙ×Yₙ matrix on the substrate. In a non-limiting example provided in FIGS. 5A-5D, three masks with square openings are used to generate a 105×105 regions on an array with 11025 unique 3-part barcodes. FIG. 5A shows the corresponding matrix of each photomask: a 7×3 matrix for the first photomask used in the first round of ligation shown in FIG. 5B, a 5×5 matrix for the second photomask shown in FIG. 5C, and a 3×7 matrix for the third photomask shown in FIG. 5D.

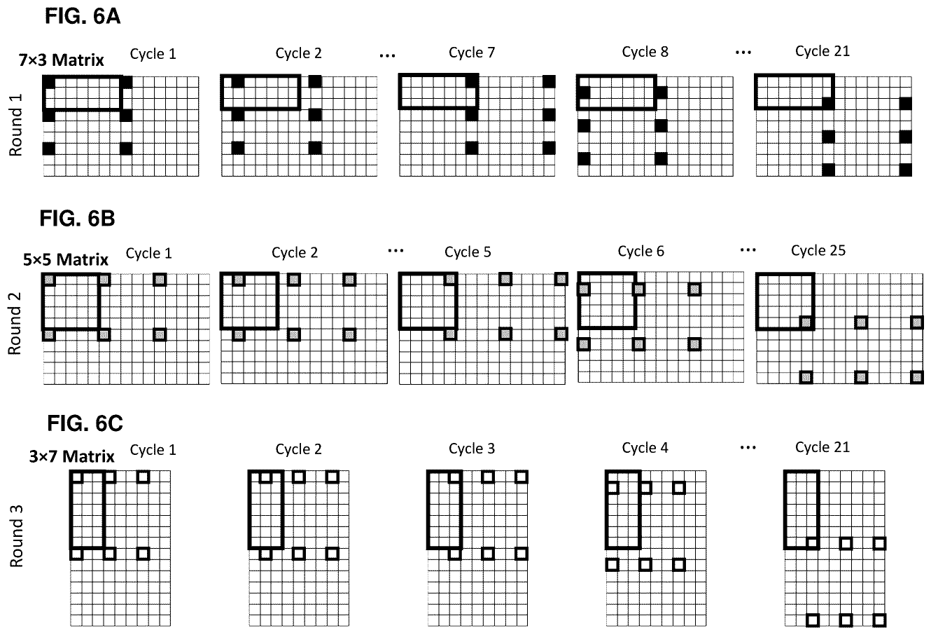

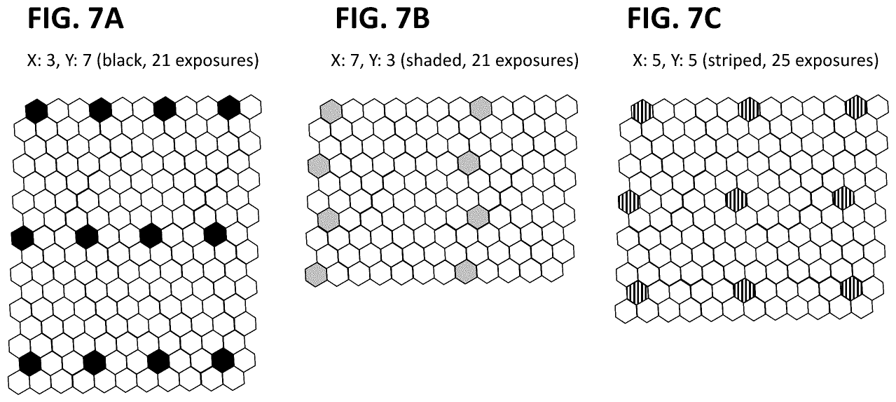

The first photomask in FIG. 5B may be translated and subjected to 7×3=21 exposures to cover the entire substrate, as shown in FIG. 6A. FIG. 6A is a schematic diagram illustrating an exemplary method of translating the first photomask in Round 1. From Cycle 1 to Cycle 7, the first photomask is horizontally translated to the right by 1 column per cycle for 7 times to cover all regions in every first of 3 rows. From Cycle 8, the mask is translated to expose regions in every second and third of 3 rows, and all regions are exposed to cover the entire substrate as of Cycle 21. Similarly, FIGS. 6B and 6C demonstrate translation of the second mask (in FIG. 5C) for 5×5=25 cycles resulting in 25 exposures, and the third mask (in FIG. 5D) for 3×7=21 cycles resulting in 21 exposures to cover the entire substrate. Hexagonal arrays may also be generated similarly as shown in FIGS. 7A-7C.

Similarly, for the 1001×1001 array with 1002001 unique barcodes, three rounds of ligation using 91, 121, and 91 oligonucleotides could be performed, with the three sparse periodic masks having the following periods in X (columns) and Y (rows): X: 13, Y: 7 (91 exposures); X: 11, Y: 11 (121 exposures); and X: 7, Y: 13 (91 exposures).

In some embodiments, maximum diversity of barcode sequence in the methods illustrated by FIGS. 5A-7C (maximum diversity=Πᵢ₌₁ⁿXᵢYᵢ) may be achieved by using photomasks wherein X₁, X₂, . . . . Xₙ do not have a common divider, and Y₁, Y₂, . . . Yₙ do not have a common divider. In some embodiments such as illustrated in FIGS. 5A-5C, X₁, Y₁, X₂, Y₂, X₃, and Y₃ are prime numbers.

Masks comprising openings of different sizes may also be used to generate the in situ array described herein. In yet another aspect, disclosed herein is a method for providing an array, comprising: (a) irradiating a substrate through a first photomask comprising an opening corresponding to a region of a plurality of regions on the substrate, wherein a first oligonucleotide of at least four nucleotides in length is attached to oligonucleotide molecules in the region to generate extended oligonucleotide molecules, wherein multiple cycles of the irradiation and oligonucleotide attachment are performed, one cycle for each of the plurality of regions, by translating the first photomask across the substrate until all regions have received the first oligonucleotide; and (b) irradiating the substrate through a second photomask comprising multiple openings corresponding to a set of sub-regions each of which is in one of the regions, wherein a second oligonucleotide of at least four nucleotides in length is attached to the extended oligonucleotide molecules in the set of sub-regions to generate further extended oligonucleotide molecules, wherein multiple cycles of the irradiation and oligonucleotide attachment are performed, one cycle for each set of sub-regions, by translating the second photomask across the substrate until all sub-regions of all regions have received the second oligonucleotide, thereby providing on the substrate an array comprising oligonucleotide molecules. In some embodiments, the method further comprises (c) irradiating the substrate through a third photomask comprising multiple openings corresponding to a set of sub-sub-regions each of which is in one of the sub-regions, wherein a third oligonucleotide of at least four nucleotides in length is attached to the further extended oligonucleotide molecules in the set of sub-sub-regions to generate even further extended oligonucleotide molecules, wherein multiple cycles of the irradiation and oligonucleotide attachment are performed, one cycle for each set of sub-sub-regions, by translating the third photomask across the substrate until all sub-sub-regions of all sub-regions of all regions have received the third oligonucleotide.

In any of the preceding embodiments, the irradiating steps can be performed in any suitable order. For instance, in Round 1, the regions can be irradiated and oligonucleotide molecules therein can receive Round 1 barcodes; in Round 2, the sub-regions can be irradiated and oligonucleotide molecules therein can receive Round 2 barcodes, thereby generating oligonucleotide molecules each comprising a Round 1 barcode and a Round 2 barcode; and in Round 3, the sub-sub-regions can be irradiated and oligonucleotide molecules therein can receive Round 3 barcodes, thereby generating oligonucleotide molecules each comprising a Round 1 barcode, a Round 2 barcode, and a Round 3 barcode.

In some embodiments, in a set of three masks, the first photomask may comprise openings corresponding to one or more regions on the substrate, the second photomask may comprise openings corresponding to one or more smaller, sub-regions relative to the regions corresponding to the openings on the first photomask, and the third photomask may comprise openings corresponding to one or more even smaller, sub-sub-regions relative to the regions corresponding to the openings on the first photomask. It should be appreciated, however, that the masks may be used in any order, and do not have to follow any specific order. FIGS. 9A-9C are schematic diagrams illustrating an exemplary method of the in situ array generation described herein. FIGS. 9A-9C shows three rounds of ligation on a substrate. In round 1 (FIG. 9A), a first photomask comprising openings corresponding to a 2×7 region is translated to cover the entire substrate. In round 2 (FIG. 9B), a second photomask comprising openings that are the size of 6×3=18 of the 2×7 regions in FIG. 9A (opening size: 6×3×2×7=252) is translated to cover the entire substrate. In round 3 (FIG. 9C), a third photomask comprising openings that are the size of 6×3=18 of the 6×3×2×7 regions in FIG. 9B is translated to cover the entire substrate.

FIGS. 10A-10D show an exemplary approach to the method demonstrated in FIGS. 9A-9C over 4 rounds each having 4 cycles (16 cycles in total). In Round 1, 4 square areas on the substrate are each ligated with a unique first part of the barcode (1 square per cycle, 4 cycles in total). Round 2 subdivides each square area from Round 1 into 4 square areas (a total of 16 square areas in Round 2), wherein in each subdivided square area within an area of Round 1 is ligated to a unique second part of the barcode. The subdivision may be achieved with a photomask comprising a pattern that correspond with the 4 dark green areas shown in Round 2, and translating the photomask to other square regions. The steps are repeated with a different photomask in Round 3 (a total of 64 square areas) and Round 4 (a total of 256 square areas), generating an array comprising 256 unique 4-part oligo barcodes. In some examples, the features are capable of achieving single cell scale resolution. For example, additional rounds shown in FIGS. 10A-10D may be used to pattern the substrate such that each feature receives a unique barcode, and the diameter of each feature is no more than 1 micron, no more than 2 microns, no more than 3 microns, no more than 4 microns, no more than 5 microns, no more than 6 microns, no more than 7 microns, no more than 8 microns, no more than 9 microns, or no more than 10 microns.

### V. SYSTEMS, KITS, AND METHODS OF USE

Also provided are compositions produced according to the methods described herein. These systems include combinations of photomasks and nucleic acid molecules and complexes generated from these photomasks, such as hybridization complexes, and kits and articles of manufacture (such as arrays) comprising such molecules and complexes.

In some embodiments, provided herein is a kit comprising the substrate, any one or more of the photomasks (e.g., the first, second, and/or third photomasks), any one or more of the oligonucleotides (e.g., the first, second, third, and/or fourth oligonucleotides), and/or any one or more of the splints (e.g., the first, second, third, and/or fourth splints) disclosed herein.

In some embodiments, provided herein is a system comprising the substrate comprising oligonucleotide molecules, the first photomask, the first oligonucleotide, and the first splint of any of the embodiments disclosed herein. In some aspects, provided herein is a system comprising the substrate comprising extended oligonucleotide molecules, the first photomask or the second photomask, the second oligonucleotide, and the second splint of any of the embodiments disclosed herein. In some aspects, provided herein is a system comprising the substrate comprising further extended oligonucleotide molecules, the third photomask, the third oligonucleotide, and the third splint of any of the embodiments disclosed herein. In some aspects, provided herein is a system comprising the substrate comprising even further extended oligonucleotide molecules, a fourth photomask, the fourth oligonucleotide, and the fourth splint disclosed herein.

In particular embodiments, provided herein is a system comprising: (i) a substrate comprising a plurality of regions; (ii) a first photomask comprising openings that correspond to all or a subset of the plurality of regions, wherein a first oligonucleotide of at least four nucleotides in length is attached to oligonucleotide molecules in the plurality of regions to generate extended oligonucleotide molecules after irradiating a plurality of regions on the substrate through the first photomask; (iii) a second photomask comprising openings that correspond to all or a subset of the plurality of regions, wherein a second oligonucleotide of at least four nucleotides in length is attached to oligonucleotide molecules in the plurality of regions to generate extended oligonucleotide molecules after irradiating a plurality of regions on the substrate through the second photomask; (iv) a third photomask comprising openings that correspond to the sub-regions, wherein a third oligonucleotide of at least four nucleotides in length is attached to oligonucleotide molecules in the plurality of regions to generate extended oligonucleotide molecules after irradiating a plurality of regions on the substrate through the third photomask. In some embodiments, the system further comprises a photoresist. In some embodiments, the photoresist forms a photoresist layer. In some embodiments, the system comprises a plurality of regions arranged in rows and columns.

In particular embodiments, provided herein is a system comprising: (i) a substrate comprising a plurality of regions; (ii) a first photomask comprising openings that correspond to all or a subset of the plurality of regions, wherein a first oligonucleotide of at least four nucleotides in length is attached to oligonucleotide molecules in the plurality of regions to generate extended oligonucleotide molecules after irradiating a plurality of regions on the substrate through the first photomask; (iii) a second photomask comprising openings that correspond to all or a subset of the plurality of regions, wherein a second oligonucleotide of at least four nucleotides in length is attached to oligonucleotide molecules in the plurality of regions to generate extended oligonucleotide molecules after irradiating a plurality of regions on the substrate through the second photomask; (iv) a third photomask comprising openings that correspond to the sub-regions, wherein a third oligonucleotide of at least four nucleotides in length is attached to oligonucleotide molecules in the plurality of regions to generate extended oligonucleotide molecules after irradiating a plurality of regions on the substrate through the third photomask. In some embodiments, the system further comprises a photoresist. In some embodiments, the photoresist forms a photoresist layer. In some embodiments, the system comprises a plurality of regions arranged in a hexagonal array.

In particular embodiments, provided herein is a system comprising: (i) a substrate comprising a plurality of regions arranged in columns and rows; (ii) a first photomask comprising periodic openings that repeat every X₁ columns and every Y₁ rows, wherein each opening corresponds to a region within a X₁×Y₁ matrix on the substrate, and wherein a first oligonucleotide of at least four nucleotides in length is attached to oligonucleotide molecules in the plurality of regions to generate extended oligonucleotide molecules; (iii) a second photomask comprising periodic openings that repeat every X₂ columns and every Y₂ rows, wherein each opening corresponds to a region within a X₂×Y₂ matrix on the substrate, and wherein a second oligonucleotide of at least four nucleotides in length is attached to oligonucleotide molecules in the plurality of regions to generate extended oligonucleotide molecules. In particular embodiments, the system further comprises (iv) a third photomask comprising periodic openings that repeat every X₃ columns and every Y₃ rows, wherein each opening corresponds to a region within a X₃×Y₃ matrix on the substrate, and wherein a first oligonucleotide of at least four nucleotides in length is attached to oligonucleotide molecules in the plurality of regions to generate extended oligonucleotide molecules. In some embodiments, X₁, X₂, and X₃ do not have a common divider (i.e., common numerical divider), and Y₁ , Y₂, and Y₃ do not have a common divider. In some embodiments, X₁, Y₁, X₂, Y₂, X₃, and Y₃ are prime numbers. In some embodiments, the system further comprises a photoresist. In some embodiments, the photoresist forms a photoresist layer. In some embodiments, the system comprises a plurality of regions arranged in rows and columns.

In particular embodiments, provided herein is a system comprising: (i) a substrate comprising a plurality of regions arranged in columns and rows; (ii) a first photomask comprising periodic openings that repeat every X₁ columns and every Y₁ rows, wherein each opening corresponds to a region within a X₁×Y₁ matrix on the substrate, and wherein a first oligonucleotide of at least four nucleotides in length is attached to oligonucleotide molecules in the plurality of regions to generate extended oligonucleotide molecules; (iii) a second photomask comprising periodic openings that repeat every X₂ columns and every Y₂ rows, wherein each opening corresponds to a region within a X₂×Y₂ matrix on the substrate, and wherein a second oligonucleotide of at least four nucleotides in length is attached to oligonucleotide molecules in the plurality of regions to generate extended oligonucleotide molecules. In particular embodiments, the system further comprises (iv) a third photomask comprising periodic openings that repeat every X₃ columns and every Y₃ rows, wherein each opening corresponds to a region within a X₃×Y₃ matrix on the substrate, and wherein a first oligonucleotide of at least four nucleotides in length is attached to oligonucleotide molecules in the plurality of regions to generate extended oligonucleotide molecules. In some embodiments, X₁, X₂, and X₃ do not have a common divider, and Y₁, Y₂, and Y₃ do not have a common divider. In some embodiments, X₁, Y₁, X₂, Y₂ , X₃ , and Y₃ are prime numbers. In some embodiments, the system further comprises a photoresist. In some embodiments, the photoresist forms a photoresist layer. In some embodiments, the system comprises a plurality of regions arranged in hexagonal arrays.

In particular embodiments, provided herein is a system comprising: (i) a substrate comprising a plurality of regions; (ii) a first photomask comprising openings that correspond a region of a plurality of regions on the substrate, wherein a first oligonucleotide of at least four nucleotides in length is attached to oligonucleotide molecules in the plurality of regions to generate extended oligonucleotide molecules after irradiating a plurality of regions on the substrate through the first photomask; (iii) a second photomask comprising openings that correspond to a set of sub-regions each of which is in one of the regions, wherein a second oligonucleotide of at least four nucleotides in length is attached to oligonucleotide molecules in the plurality of regions to generate extended oligonucleotide molecules after irradiating a plurality of regions on the substrate through the second photomask; (iv) a third photomask comprising openings that correspond to a set of sub-sub-regions each of which is in one of the sub-regions, wherein a third oligonucleotide of at least four nucleotides in length is attached to oligonucleotide molecules in the plurality of regions to generate extended oligonucleotide molecules after irradiating a plurality of regions on the substrate through the third photomask. In some embodiments, the system further comprises a photoresist. In some embodiments, the photoresist forms a photoresist layer. In some embodiments, the system comprises a plurality of regions arranged in rows and columns.

In particular embodiments, provided herein is a system comprising: (i) a substrate comprising a plurality of regions; (ii) a first photomask comprising openings that correspond a region of a plurality of regions on the substrate, wherein a first oligonucleotide of at least four nucleotides in length is attached to oligonucleotide molecules in the plurality of regions to generate extended oligonucleotide molecules after irradiating a plurality of regions on the substrate through the first photomask; (iii) a second photomask comprising openings that correspond to a set of sub-regions each of which is in one of the regions, wherein a second oligonucleotide of at least four nucleotides in length is attached to oligonucleotide molecules in the plurality of regions to generate extended oligonucleotide molecules after irradiating a plurality of regions on the substrate through the second photomask; (iv) a third photomask comprising openings that correspond to a set of sub-sub-regions each of which is in one of the sub-regions, wherein a third oligonucleotide of at least four nucleotides in length is attached to oligonucleotide molecules in the plurality of regions to generate extended oligonucleotide molecules after irradiating a plurality of regions on the substrate through the third photomask. In some embodiments, the system further comprises a photoresist. In some embodiments, the photoresist forms a photoresist layer. In some embodiments, the system comprises a plurality of regions arranged in a hexagonal array.

Also provided herein are arrays comprising any one or more of the molecules, complexes, and/or compositions disclosed herein. Typically, an array includes at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g. as a spot on the surface of the substrate. The number of distinct nucleic acid sequences, and hence spots or similar structures, present on the array may vary, but is generally at least, usually at least 5 and more usually at least 10, where the number of different spots on the array may be as a high as 50, 100, 500, 1000, 10,000, 1,000,000, 10,000,000 or higher, depending on the intended use of the array. The spots of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like. The density of spots present on the array surface may vary, but is generally at least about 10 and usually at least about 100 spots/cm², where the density may be as high as 10⁶ or higher, or about 10⁵ spots/cm². In other embodiments, the polymeric sequences are not arranged in the form of distinct spots, but may be positioned on the surface such that there is substantially no space separating one polymer sequence/feature from another. The density of nucleic acids within an individual feature on the array may be as high as 1,000, 10,000, 25,000, 50,000, 100,000, 500,000, 1,000,000, or higher per square micron depending on the intended use of the array.

In some embodiments, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini, e.g. the 3′ or 5′ terminus.

Arrays can be used to measure large numbers of analytes and/or proxies thereof simultaneously. In some embodiments, oligonucleotides are used, at least in part, to create an array. For example, one or more copies of a single species of oligonucleotide (e.g., capture probe) can correspond to or be directly or indirectly attached to a given feature in the array. In some embodiments, a given feature in the array includes two or more species of oligonucleotides (e.g., capture probes). In some embodiments, the two or more species of oligonucleotides (e.g., capture probes) attached directly or indirectly to a given feature on the array include a common (e.g., identical) spatial barcode.

In some embodiments, an array can include a capture probe attached directly or indirectly to the substrate. The capture probe can include a capture domain (e.g., a nucleotide sequence) that can specifically bind (e.g., hybridize) to a target analyte (e.g., mRNA, DNA, or protein) or a proxy thereof (e.g., a ligation product obtained from templated ligation of a probe pair) within a sample. In some embodiments, the binding (e.g., hybridization) of the capture probe to the target or a proxy thereof can be detected and quantified by detection of a visual signal, e.g., a fluorophore, a heavy metal (e.g., silver ion), or chemiluminescent label, which has been incorporated into the target. In some embodiments, the intensity of the visual signal correlates with the relative abundance of each analyte in the biological sample. Since an array can contain thousands or millions of capture probes (or more), an array can interrogate many analytes or proxies thereof in parallel. In some embodiments, the binding (e.g., hybridization) of the capture probe to the target can be detected and quantified by creation of a molecule (e.g., cDNA from captured mRNA generated using reverse transcription) that is removed from the array, and sequenced.

Kits for use in analyte detection assays are provided. In some embodiments, the kit at least includes an array disclosed herein. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the subject array assay devices for carrying out an array based assay. The instructions may be printed on a substrate, such as paper or plastic, etc. As such, the instructions may be present in the kits as a package insert, in the labeling of the container of the kit or components thereof (i.e., associated with the packaging or sub-packaging) etc.

The subject arrays find use in a variety of different applications, where such applications are generally analyte detection applications in which the presence of a particular analyte or a proxy thereof in a given sample is detected at least qualitatively, if not quantitatively. Protocols for carrying out such assays are well known to those of skill in the art and need not be described in great detail here. Generally, the sample suspected of comprising the analyte of interest analyte (or a proxy thereof) is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc., and/or through sequencing of one or more components of the binding complex or a product thereof. The presence of the analyte or a proxy thereof in the sample is then deduced from the detection of binding complexes on the substrate surface, or sequence detection and/or analysis (e.g., by sequencing) on molecules indicative of the formation of the binding complex. In some embodiments, RNA molecules (e.g., mRNA) from a sample are captured by oligonucleotides (e.g., probes comprising a barcode and a poly(dT) sequence) on an array prepared by a method disclosed herein, cDNA molecules are generated via reverse transcription of the captured RNA molecules, and the cDNA molecules (e.g., a first strand cDNA) or portions or products (e.g., a second strand cDNA synthesized using a template switching oligonucleotide) thereof can be separated from the array and sequenced. Sequencing data obtained from molecules prepared on the array can be used to deduce the presence/absence or an amount of the RNA molecules in the sample.

Specific analyte detection applications of interest include hybridization assays in which the nucleic acid arrays of the subject invention are employed. In these assays, a sample of target nucleic acids or a tissue section is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The formation and/or presence of hybridized complexes is then detected, e.g., by analyzing molecules that are generated following the formation of the hybridized complexes, such as cDNA or a second strand generated from an RNA captured on the array. Specific hybridization assays of interest which may be practiced using the subject arrays include: gene discovery assays, differential gene expression analysis assays; nucleic acid sequencing assays, and the like.

### A. Spatial Analysis

In particular embodiments, provided herein are kits and compositions for spatial array-based analysis of biological samples. Array-based spatial analysis methods involve the transfer of one or more analytes or proxies thereof from a biological sample to an array of features on a substrate, where each feature is associated with a unique spatial location on the array. Subsequent analysis of the transferred analytes or proxies thereof includes determining the identity of the analytes and the spatial location of each analyte within the biological sample. The spatial location of each analyte within the biological sample is determined based on the feature to which each analyte is bound on the array, and the feature's relative spatial location within the array. In some embodiments, the array of features on a substrate comprise a spatial barcode that corresponds to the feature's relative spatial location within the array. Each spatial barcode of a feature may further comprise a fluorophore, to create a fluorescent hybridization array. A feature may comprise UMIs that are generally unique per nucleic acid molecule in the feature—this is so the number of unique molecules can be estimated, as opposed to an artifact in experiments or PCR amplification bias that drives amplification of smaller, specific nucleic acid sequences.

In particular embodiments, the kits and compositions for spatial array-based analysis provide for the detection of differences in an analyte level (e.g., gene and/or protein expression) within different cells in a tissue of a mammal or within a single cell from a mammal. For example, the kits and compositions can be used to detect the differences in analyte levels (e.g., gene and/or protein expression) within different cells in histological slide samples (e.g., intact tissue section), the data from which can be reassembled to generate a three-dimensional map of analyte levels (e.g., gene and/or protein expression) of a tissue sample obtained from a mammal, e.g., with a degree of spatial resolution (e.g., single-cell scale resolution).

In some embodiments, an array generated using a method disclosed herein can be used in array-based spatial analysis methods which involve the transfer of one or more analytes from a biological sample to an array of features on a substrate, each of which is associated with a unique spatial location on the array. Subsequent analysis of the transferred analytes includes determining the identity of the analytes and the spatial location of each analyte within the sample. The spatial location of each analyte within the sample is determined based on the feature to which each analyte is bound in the array, and the feature's relative spatial location within the array.

There are at least two general methods to associate a spatial barcode with one or more neighboring cells, such that the spatial barcode identifies the one or more cells, and/or contents of the one or more cells, as associated with a particular spatial location. One general method is to drive target analytes out of a cell and towards the spatially-barcoded array. In some embodiments, the spatially-barcoded array populated with capture probes is contacted with a sample, and sample is permeabilized, allowing the target analyte or proxy thereof to migrate away from the sample and toward the array. The target analyte or proxies thereof interact with a capture probe on the spatially-barcoded array. Once the target analyte or proxy hybridizes/is bound to the capture probe, the sample is optionally removed from the array and the capture probes are analyzed in order to obtain spatially-resolved analyte information. Methods for performing such spatial analysis of tissue sections are known in the art and include but are not limited to those methods disclosed in U.S. Pat. Nos. 10,030,261, 11,332,790 and US Patent Pub No. 20220127672 and US Patent Pub No. 20220106632, the contents of which are herein incorporated by reference in their entireties.

Another general method is to cleave the spatially-barcoded capture probes from an array, and drive the spatially-barcoded capture probes towards and/or into or onto the sample. In some embodiments, the spatially-barcoded array populated with capture probes is contacted with a sample. The spatially-barcoded capture probes are cleaved and then interact with cells within the provided sample (See, for example, U.S. Pat. No. 11,352,659 the contents of which are herein incorporate by reference in its entirety). The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed (e.g., by sequencing) to obtain spatially-resolved information about the tagged cell.

Sample preparation may include placing the sample on a slide, fixing the sample, and/or staining the sample for imaging. The stained sample may be imaged on the array using both brightfield (to image the sample hematoxylin and eosin stain) and/or fluorescence (to image features) modalities. In some embodiments, target analytes are then released from the sample and capture probes forming the spatially-barcoded array hybridize or bind the released target analytes. The sample is then removed from the array and the capture probes cleaved from the array. The sample and array are then optionally imaged a second time in one or both modalities (brightfield and fluorescence) while the analytes are reverse transcribed into cDNA, and an amplicon library is prepared and sequenced. The two sets of images can then be spatially-overlaid in order to correlate spatially-identified sample information. When the sample and array are not imaged a second time, a spot coordinate file may be supplied. The spot coordinate file can replace the second imaging step. Further, amplicon library preparation can be performed with a unique PCR adapter and sequenced.

In some embodiments, a spatially-labelled array on a substrate is used, where capture probes labelled with spatial barcodes are clustered at areas called features. The spatially-labelled capture probes can include a cleavage domain, one or more functional sequences, a spatial barcode, a unique molecular identifier, and a capture domain. The spatially-labelled capture probes can also include a 5′ end modification for reversible attachment to the substrate. The spatially-barcoded array is contacted with a sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. The sample can be optionally removed from the array.

Adapters and assay primers can be used to allow the capture probe or the analyte capture agent to be attached to any suitable assay primers and used in any suitable assays. A capture probe that includes a spatial barcode can be attached to a bead that includes a poly(dT) sequence. A capture probe including a spatial barcode and a poly(T) sequence can be used to assay multiple biological analytes as generally described herein (e.g., the biological analyte includes a poly(A) sequence or is coupled to or otherwise is associated with an analyte capture agent comprising a poly(A) sequence as the analyte capture sequence).

The capture probes can be optionally cleaved from the array, and the captured analytes can be spatially-tagged by performing a reverse transcriptase first strand cDNA reaction. A first strand cDNA reaction can be optionally performed using template switching oligonucleotides. For example, a template switching oligonucleotide can hybridize to a poly(C) tail added to a 3′end of the cDNA by a reverse transcriptase enzyme. The original mRNA template and template switching oligonucleotide can then be denatured from the cDNA and the barcoded capture probe can then hybridize with the cDNA and a complement of the cDNA can be generated. The first strand cDNA can then be purified and collected for downstream amplification steps. The first strand cDNA can be amplified using PCR, wherein the forward and reverse primers flank the spatial barcode and target analyte regions of interest, generating a library associated with a particular spatial barcode. In some embodiments, the cDNA comprises a sequencing by synthesis (SBS) primer sequence. The library amplicons are sequenced and analyzed to decode spatial information.

In some embodiments, the sample is removed from the spatially-barcoded array and the spatially-barcoded capture probes are removed from the array for barcoded analyte amplification and library preparation. Another embodiment includes performing first strand synthesis using template switching oligonucleotides on the spatially-barcoded array without cleaving the capture probes. Once the capture probes capture the target analyte(s), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, and a-tailing, and indexing PCR steps, and then sequenced.

### VI. TERMINOLOGY

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used in this specification and the appended claims, the singular forms “a,” “an” and “the” include plural referents unless the context clearly dictates otherwise. Thus, for example, reference to “a molecule” includes a plurality of such molecules, and the like.

A sample such as a biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

The term “barcode,” comprises a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell scale resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

As used herein, the term “substrate” generally refers to a substance, structure, surface, material, means, or composition, which comprises a nonbiological, synthetic, nonliving, planar, spherical or flat surface. The substrate may include, for example and without limitation, semiconductors, synthetic metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes; silicon, silicates, glass, metals and ceramics; wood, paper, cardboard, cotton, wool, cloth, woven and nonwoven fibers, materials and fabrics; nanostructures and microstructures. The substrate may comprise an immobilization matrix such as but not limited to, insolubilized substance, solid phase, surface, layer, coating, woven or nonwoven fiber, matrix, crystal, membrane, insoluble polymer, plastic, glass, biological or biocompatible or bioerodible or biodegradable polymer or matrix, microparticle or nanoparticle. Other example may include, for example and without limitation, monolayers, bilayers, commercial membranes, resins, matrices, fibers, separation media, chromatography supports, polymers, plastics, glass, mica, gold, beads, microspheres, nanospheres, silicon, gallium arsenide, organic and inorganic metals, semiconductors, insulators, microstructures and nanostructures. Microstructures and nanostructures may include, without limitation, microminiaturized, nanometer-scale and supramolecular probes, tips, bars, pegs, plugs, rods, sleeves, wires, filaments, and tubes.

As used herein, the term “nucleic acid” generally refers to a polymer comprising one or more nucleic acid subunits or nucleotides. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double-stranded.

The term “nucleic acid sequence” or “nucleotide sequence” as used herein generally refers to nucleic acid molecules with a given sequence of nucleotides, of which it may be desired to know the presence or amount. The nucleotide sequence can comprise ribonucleic acid (RNA) or DNA, or a sequence derived from RNA or DNA. Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 20, 50, 100, 200, 300, 400, 500, 600, 700, 800, 1000, 1200, 1500, 2000, 5000, 10000 or more than 10000 nucleotides in length, or at least about 20, 100, 200, 300, 400, 500, 600, 700, 800, 1000, 1200, 1500, 2000, 5000, 10000 nucleotides in length.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (i.e., can be oligoribonucleotides) and/or deoxyribonucleotide monomers (i.e., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

As used herein, the term “adjacent” or “adjacent to,” includes “next to,” “adjoining,” and “abutting.” In one example, a first location is adjacent to a second location when the first location is in direct contact and shares a common border with the second location and there is no space between the two locations. In some cases, the adjacent is not diagonally adjacent.

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “proximity ligation” is a method of ligating two (or more) nucleic acid sequences that are in proximity with each other through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference).

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation

As used herein, the term “splint” is an oligonucleotide that, when hybridized to other polynucleotides, acts as a “splint” to position the polynucleotides next to one another so that they can be ligated together. In some embodiments, the splint is DNA or RNA. The splint can include a nucleotide sequence that is partially complimentary to nucleotide sequences from two or more different oligonucleotides. In some embodiments, the splint assists in ligating a “donor” oligonucleotide and an “acceptor” oligonucleotide. In general, an RNA ligase, a DNA ligase, or another other variety of ligase is used to ligate two nucleotide sequences together

In some embodiments, the splint is between 6 and 50 nucleotides in length, e.g., between 6 and 45, 6 and 40, 6 and 35, 6 and 30, 6 and 25, or 6 and 20 nucleotides in length. In some embodiments, the splint is between 10 and 50 nucleotides in length, e.g., between 10 and 45, 10 and 10 and 35, 10 and 30, 10 and 25, or 10 and 20 nucleotides in length. In some embodiments, the splint is between 15 and 50, 15 and 45, 15 and 40, 15 and 35, 15 and 30, or 15 and 25 nucleotides in length.

A “feature” is an entity that acts as a support or repository for various molecular entities used in sample analysis. In some embodiments, some or all of the features in an array are functionalized for analyte capture. In some embodiments, functionalized features include one or more capture probe(s). Examples of features include, but are not limited to, a bead, a spot of any two- or three-dimensional geometry (e.g., an ink jet spot, a masked spot, a square on a grid), a well, and a hydrogel pad. In some embodiments, features are directly or indirectly attached or fixed to a substrate. In some embodiments, the features are not directly or indirectly attached or fixed to a substrate, but instead, for example, are disposed within an enclosed or partially enclosed three dimensional space (e.g., wells or divots).

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “template” as used herein generally refers to individual polynucleotide molecules from which another nucleic acid, including a complementary nucleic acid strand, can be synthesized by a nucleic acid polymerase. In addition, the template can be one or both strands of the polynucleotides that are capable of acting as templates for template-dependent nucleic acid polymerization catalyzed by the nucleic acid polymerase. Use of this term should not be taken as limiting the scope of the present disclosure to polynucleotides which are actually used as templates in a subsequent enzyme-catalyzed polymerization reaction. The template can be an RNA or DNA. The template can be cDNA corresponding to an RNA sequence. The template can be DNA.

As used herein, “amplification” of a template nucleic acid generally refers to a process of creating (e.g., in vitro) nucleic acid strands that are identical or complementary to at least a portion of a template nucleic acid sequence, or a universal or tag sequence that serves as a surrogate for the template nucleic acid sequence, all of which are only made if the template nucleic acid is present in a sample. Typically, nucleic acid amplification uses one or more nucleic acid polymerase and/or transcriptase enzymes to produce multiple copies of a template nucleic acid or fragments thereof, or of a sequence complementary to the template nucleic acid or fragments thereof. In vitro nucleic acid amplification techniques are may include transcription-associated amplification methods, such as Transcription-Mediated Amplification (TMA) or Nucleic Acid Sequence-Based Amplification (NASBA), and other methods such as Polymerase Chain Reaction (PCR), Reverse Transcriptase-PCR (RT-PCR), Replicase Mediated Amplification, and Ligase Chain Reaction (LCR).

In addition to those above, a wide variety of other features can be used to form the arrays described herein. For example, in some embodiments, features that are formed from polymers and/or biopolymers that are jet printed, screen printed, or electrostatically deposited on a substrate can be used to form arrays.

### EXAMPLE

The following example is included for illustrative purposes only and is not intended to limit the scope of the invention.

### Example 1: Analyzing Mouse Brain Tissue Using a Spatial Barcode Array

This example demonstrates an application of an array generated using a minimal mask scheme photo-hybridization/ligation method, described herein, for analyzing a mouse brain tissue section.

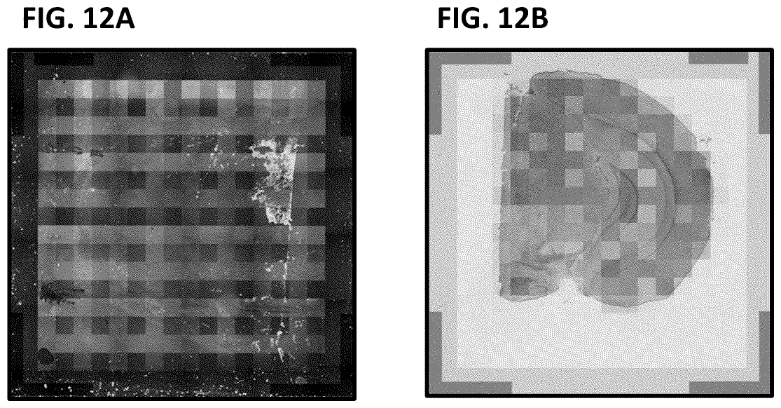

FIG. 12A shows an exemplary spatial array generated using a combinatorial barcoding approach as shown in FIGS. 10A-10D. 256 square areas in the 7×7 mm array area were generated over 4 rounds of photo-hybridization/ligation (4 cycles per round, 16 cycles in total). Using a scheme similar to that shown in FIGS. 10A-10D, features within each of the 4 square areas in Round 1 received barcode Part 1, features within each of the 16 smaller square areas in Round 2 received barcode Part 2, features within each of the 64 even smaller square areas in Round 3 received barcode Part 3, and features within each of the final 256 square areas in Round 4 received barcode Part 4 of a spatial barcode. Exemplary barcode sequences after Round 4 are shown in FIG. 11. A spatial array comprising 256 combinatorially generated 4-part spatial barcodes was generated, where each of the 256 spatial barcodes was unique to the square area but shared by the feature and the oligonucleotide probes within the feature. Fluorescently labelled probes for the 256 unique spatial barcodes were applied to the array to generate the fluorescent image in FIG. 12A.

A mouse brain tissue section (10 micron thickness) was deposited on the spatial array and processed as follows. The tissue section was permeabilized, and mRNA molecules from the tissue section were captured on the 256 feature spatial array via polyT sequences of the capture domains in the oligonucleotide probes on the spatial array. After the mRNAs hybridized to the capture domains of the oligonucleotides of the spatial array, first strand cDNAs were created using reverse transcription of the captured mRNAs. Second strand synthesis was performed via template switching to generate second strand cDNAs, which were denatured from the spatial array and sequenced. The second strand cDNAs contained nucleotide sequences complementary to the spatial barcodes and UMIs of the oligonucleotide probes on the array, allowing the sequencing reads to be mapped back to one of the 256 features on the spatial array.

The tissue section was stained using H&E (hematoxylin and eosin) and imaged. The sequencing reads were refined to remove duplicate UMI's allowing for accurate quantitation of analytes (e.g., mRNA transcripts) in the tissue section. UMIs by feature (i.e., spatial barcode) were overlaid with the H&E image in FIG. 12B, showing that the mRNA transcript gene expression profile obtained using the 256 combinatorially generated 4-part spatial array generally matched the expected transcript profile of mouse brain tissue. These results suggest that expression profiles for individual transcripts can be analyzed with an array generated using a photo-hybridization/ligation method disclosed herein, and combined with the information obtained from the tissue sections through staining (e.g., H&E or immunofluorescent (IF) stain).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-87. (canceled)

88. A method for providing an array, comprising:
(a) irradiating a plurality of regions on a substrate through a first photomask comprising openings that correspond to all or a subset of the plurality of regions,
wherein a first oligonucleotide of at least four nucleotides in length is attached to oligonucleotide molecules in the plurality of regions to generate extended oligonucleotide molecules;
(b) irradiating the plurality of regions through the first photomask rotated relative to the substrate, or through a second photomask comprising openings that correspond to all or a subset of the plurality of regions,
wherein a second oligonucleotide of at least four nucleotides in length is attached to the extended oligonucleotide molecules in the plurality of regions to generate further extended oligonucleotide molecules; and
(c) irradiating sub-regions of the plurality of regions through a third photomask comprising openings that correspond to the sub-regions,
wherein a third oligonucleotide of at least four nucleotides in length is attached to the further extended oligonucleotide molecules in the sub-regions,
thereby providing on the substrate an array comprising oligonucleotide molecules.

89. The method of claim 88, wherein the plurality of regions are arranged in rows and columns on the substrate, and the first photomask and/or the second photomask comprises openings that correspond to one or more of the rows or one or more of the columns.

90. The method of claim 89, wherein the rows are parallel to each other and/or the columns are parallel to each other.

91. The method of claim 89, wherein the angle between an intersecting row and column is 90 degrees.

92. The method of claim 88, wherein the plurality of regions are arranged in a square array or a hexagonal array.

93. The method of claim 89, wherein (a) comprises multiple cycles of irradiation and oligonucleotide attachment, and in one of the cycles the substrate is irradiated through the first photomask at a subset of the rows or a subset of the columns.

94. The method of claim 93, wherein the method comprises, after irradiation and oligonucleotide attachment to the oligonucleotide molecules in a first column, translating the first photomask such that the openings in the first photomask correspond to a second column of the plurality of regions.

95. The method of claim 94, wherein the first and second columns do not overlap.

96. The method of claim 94, comprising translating the first photomask and performing multiple cycles of irradiation and oligonucleotide attachment until all columns have received the first oligonucleotide.

97. The method of claim 88, wherein the first oligonucleotide comprises a sequence that hybridizes to a first splint which in turn hybridizes to the oligonucleotide molecules in the plurality of regions, and wherein the first oligonucleotide is ligated to the oligonucleotide molecules using the first splint as template to generate the extended oligonucleotide molecules.

98. The method of claim 88, wherein the first oligonucleotide comprises a sequence that hybridizes to a second splint which in turn hybridizes to the second oligonucleotide, and wherein the second oligonucleotide is ligated to the extended oligonucleotide molecules using the second splint as template to generate the further extended oligonucleotide molecules.

99. The method of claim 88, wherein the first oligonucleotide comprises a first barcode sequence that is different for different columns.

100. The method of claim 88, wherein the openings in the first photomask correspond to a column of the plurality of regions and wherein (b) comprises rotating the first photomask to irradiate a first row of the plurality of regions through the openings in the first photomask, wherein the second oligonucleotide is attached to the extended oligonucleotide molecules in the first row.

101. The method of claim 100, wherein the method comprises, after irradiation and oligonucleotide attachment to the extended oligonucleotide molecules in the first row, translating the first photomask such that the openings in the first photomask correspond to a second row of the plurality of regions.

102. The method of claim 101, wherein the first and second rows do not overlap.

103. The method of claim 101, comprising translating the first photomask and performing multiple cycles of irradiation and oligonucleotide attachment until all rows have received the second oligonucleotide.

104. The method of claim 88, wherein the substrate comprises a lawn of universal oligonucleotide molecules prior to irradiation.

105. The method of claim 88, wherein any one or more of the irradiating steps comprise photolithography using a photoresist.

106. The method of claim 88, wherein oligonucleotide molecules in the provided array are located in regions which are between 1 micron and 5 microns in diameter.

107. The method of claim 88, wherein the substrate is a chip, a wafer, a die, or a slide and the oligonucleotide molecules, the extended oligonucleotide molecules, the further extended oligonucleotide molecules, and the even further extended oligonucleotide molecules are provided or generated in the absence of a cell or tissue sample on the substrate.
